(19) United States
(12) Patent Application Publication
(10) Pub. No.: US 2026/0360474 A1
(43) Pub. Date: Nov. 12, 2026

(54) METHODS, SYSTEMS, AND DEVICES FOR ANALYZING DATA FROM BIOLOGICAL SAMPLE ASSAYS

(71) Applicant: LIFE TECHNOLOGIES CORPORATION, Carlsbad, CA (US)

(72) Inventors: David LIU, Sunnyvale, CA (US); Noah G. ELDER, Lafayette, CA (US); Chris THOMAS, San Mateo, CA (US); Carmen GJERSTAD, Millbrae, CA (US)

(21) Appl. No.: 19/720,723

(22) Filed: Jun. 25, 2026

Related U.S. Application Data
(63) Continuation of application No. PCT/US2025/010276, filed on Jan. 3, 2025.
(60) Provisional application No. 63/617,990, filed on Jan. 5, 2024.

(51) Int. Cl. G16B 45/00; C12Q 1/6851; G16B 25/20

(52) U.S. Cl. CPC G16B 45/00; G16B 25/20; C12Q 1/6851

(57) ABSTRACT

Systems, methods, and computer readable media with instructions for automated thresholding of dPCR assay data include generating a histogram from emission data collected from reaction sites of a dPCR assay, the histogram comprising a plurality of bins representing a number of reaction sites at different levels of emission signal; based on a cluster of high concentration bins (representing data that is positive or negative for amplification product based on emission signal level), identifying bins having low concentration based on a number of reaction sites per bin being less than or equal to a predetermined amount, and then setting a threshold based on lowest or highest emission signal levels in the low concentration bins.

### CROSS-REFERENCE TO RELATED APPLICATIONS

This application is a continuation of PCT application No. PCT/US2025/010276, filed Jan. 3, 2025, which claims benefit of U.S. Provisional Application No. 63/617,990, filed Jan. 5, 2024. The entire contents of the aforementioned applications are incorporated by reference herein.

### TECHNICAL FIELD

This disclosure relates to biological sample analysis techniques. More specifically, aspects of the disclosure relate to determining data thresholds for use in analyzing data from digital polymerase chain reaction (dPCR) assays.

### INTRODUCTION

Nucleic acid detection assays can be conducted by adding a sample that is suspected of including one or more target nucleic acids to a reaction mixture. The reaction mixture can include one or more probes carrying detectable labels each designed to interact with one or more different target nucleic acids and generate an emission signal, via the detectable labels, which corresponds to the amount of target nucleic acid in the reaction mixture. Subjecting the sample and reaction mixture to an amplification reaction results in an amplified product of the target nucleic acid (amplicons (copies) of the target nucleic acid) when present in the starting sample. By detecting the signal from the detectable labels of the probe which interacted with the amplified product, the initial amount of target nucleic acid can be determined.

Digital polymerase chain reaction (dPCR) is a technique that allows amplification of a single nucleic template strand from a diluted sample, thus generating amplicons that are exclusively derived from one template and can be detected with different detectable labels (e.g., fluorescent molecules). It can be used in various applications, including, for example, to determine the concentration of target nucleic acid in a sample, discriminate different alleles (e.g., wild type vs. mutant or paternal vs. maternal alleles) in a sample, detect rare alleles in a sample, among others. The basic premise of the technique is to divide a large sample into a number of smaller subvolumes (segmented volumes), whereby the subvolumes contain on average a single copy of a target. Then, by detecting the detectable label associated with the generation of amplified product and counting the number of the subvolumes containing detectable amounts of the detectable label, the starting copy number (concentration) of the target in the starting volume of sample can be determined.

Thus, in dPCR, amplification is detected from reaction volumes with nucleic acid template and no amplification is detected in reaction volumes lacking a nucleic acid template. More specifically, emission signals from the detectable labels used during the dPCR assay are detected from reaction sites containing the reaction volumes with nucleic acid template. But because a background level of emission signal can occur and be detected even in reaction volumes lacking the template (and thus lacking amplified product of the same), a threshold signal level is determined and used to compare detected emission signal so as to make a determination as to whether a reaction volume is positive (i.e., contains amplification product of the target nucleic acid) or negative (i.e., does not contain amplification product of the target). This process of making a determination of a reaction volume as positive or negative is sometimes referred to as a “call” for each reaction volume. As used herein, reaction volume is used interchangeable with reaction partition and can encompass a particle or other substrate at which a nucleic acid template is linked or bound.

Challenges associated with dPCR assays thus include determining the threshold level of detectable emission signal to accurately make the call of a reaction volume as being positive or negative. Further, due to the vast amount of data generated from dPCR assays, challenges can exist with user-based techniques for determining thresholds.

A need therefore exists for a robust approach to determining emission signal level thresholds in analysis of dPCR assay data that enhances accuracy, efficiency in data analysis, and allows for an automated approach alleviating user input and potential subjectivity.

### SUMMARY

Embodiments of the present disclosure may solve one or more of the above-mentioned problems and/or may demonstrate one or more of the above-mentioned desirable features. Other features and/or advantages may become apparent from the description that follows.

In various implementations, the present disclosure contemplates a method for analyzing emission signal data from a digital polymerase chain reaction (dPCR) assay and includes generating a histogram from emission data collected from reaction sites of a dPCR assay, the histogram may include a plurality of bins representing a number of reaction sites at different levels of emission signal. The method also includes identifying a first subset of the plurality of bins, the first subset may include bins having low concentration based on a number of reaction sites per bin being less than or equal to a predetermined amount. The method also includes identifying a first bin in the first subset of bins that corresponds to a lowest level of emission signal. The method also includes setting a first threshold estimate of emission signal level at the lowest level of emission signal of the identified first bin. The method also includes identifying a second subset of the plurality of bins adjacent to and over a range of emission signal level higher than the first threshold estimate of emission signal level. The method also includes identifying a second bin in the second subset of bins that corresponds to a highest level of emission signal. The method also includes setting a second threshold estimate of emission signal level at the highest level of emission signal of the identified second bin. The method also includes comparing the population of data below the first threshold estimate to the population of data below the second threshold estimate. The method also includes based on the comparing, setting a threshold level for classification of the emission signal data as either the first threshold estimate or the second threshold estimate. The method also includes for a reaction site, classifying emission signal data from the reaction site as positive for amplification of target from the dPCR assay if the level of emission signal corresponding to the reaction site is above the threshold level or classifying the emission signal data as negative for amplification of target from the dPCR assay if the level of emission signal corresponding to the reaction site is below the threshold level.

Additional implementations may include one or more of the following. The method may include setting a bin size for each of the plurality of bins. Setting the bin size may include setting the bin size based on expected emission signal level associated with a reaction volume being positive for amplification product. Setting the bin size may include setting the bin size such that each bin is a predetermined percentage of the expected emission signal level. The predetermined percentage can range from 0.1% to 10%. The expected emission signal level is based on a positive control signal. The comparing may include comparing a standard deviation of the population below the first threshold estimate to a standard deviation of the population below the second threshold estimate, and setting the threshold level for classification may include: setting the threshold level to the first threshold estimate if the standard deviation of the population below the second threshold estimate is greater than three times the standard deviation of the population below the first threshold estimate, or setting the threshold level to the second threshold estimate if standard deviation of the population below the second threshold estimate is less than three times the standard deviation of the population below the first threshold estimate.

The plurality of bins from which the first subset is identified includes bins in a range from a defined minimum level of emission signal to a defined maximum level of emission signal. The defined minimum level of emission signal is a level corresponding to a range of from 0% to 40% quantile of an entire population of emission data. The predetermined minimum level of emission signal is a level corresponding to a 5% quantile of the entire population of emission data. The predetermined maximum level of emission signal is a level corresponding to a range of 60% or more of the quantile of the entire population of emission data. The predetermined maximum level of emission signal is a level corresponding to a 95% quantile of the entire population of an emission data. Identifying the second subset of bins may include identifying the second subset of bins from bins in a range corresponding to a level of emission signal ranging from first threshold estimate of emission signal level to a defined maximum level of emission signal level. The defined maximum level of emission signal is determined by: calculating a first value equal to two times a difference of a 95% quantile of the population of emission data below the first threshold estimate and a 5% quantile of the population of emission data below the first threshold estimate; calculating a second value equal to ⅙th of an expected positive population of emission data; and adding a highest of the first value or the second value to the first threshold estimate of emission signal level to determine the defined maximum level of emission signal. The first subset may include bins having a number of reaction sites per bin less than or equal to 0.05% to 10% of the total number of data points in all the bins of the histogram. The first subset may include bins having a number of reaction sites per bin less than or equal to 0.1% of the total number of data points in all the bins of the histogram. Setting the bin size may include setting the bin size based on applying Sturges formula. The method may include setting a soft threshold based on a positive control signal. The method may include determining one or more attributes of the emission data based on the soft threshold and using the one or more attributes to adjust one or more of a range of data for the first subset of data and/or a bin size for the bin. The method may include: comparing the threshold level to a positive control signal value; and on condition of the threshold level being greater than the positive control signal by a determined amount, one or both of recalculating the threshold or outputting feedback indicative of an error, or on condition of the threshold level no being greater than the positive control signal by the determined amount, using the threshold level for classifying the emission data.

In accordance with various other implementations the present disclosure contemplates a method for analyzing emission signal data from a digital PCR (dPCR) assay including generating a histogram from emission data collected from reaction sites of a dPCR assay, the histogram may include a plurality of bins representing a number of reaction sites at different levels of emission signal. The method also includes identifying a continuous range of bins, where each bin in the continuous range has a number of reaction sites per bin less than or equal to a predetermined low concentration of number of reaction sites per bin. The method also includes selecting a bin from the continuous range of bins. The method also includes setting the threshold emission signal level as an emission signal level in the selected bin. The method also includes for a reaction site, classifying emission signal data from the reaction site as positive for amplification of target from the dPCR assay if a level of emission signal corresponding to the reaction site is above the threshold level or classifying the emission signal data from the reaction site as negative for amplification of the dPCR assay if the level of emission signal corresponding to the reaction site is below the threshold level.

Additional implementations may include one or more of the following. Selecting the bin from the continuous range of bins may include selecting a bin corresponding to a highest level of emission signal in the continuous range of bins. Selecting the bin may include selecting a bin adjacent to a cluster of bins corresponding to a range of emission signal levels indicating positive for amplification product from the dPCR assay. The first subset may include bins having a number of reaction sites per bin less than or equal to 0.05% to 10% of the total number of data points in all the bins of the histogram. The continuous range of bins may include bins having a number of reaction sites per bin less than or equal to 0.1% of the total number of data points in all the bins of the histogram. Selecting the bin from the continuous range of bins may include selecting a bin corresponding to a lowest level of emission signal in the continuous range of bins. Selecting the bin may include selecting a bin adjacent to a cluster of bins corresponding to a range of emission signal levels indicating negative for amplification product from the dPCR assay. Selecting the bin and setting the threshold may include selecting one of the first or second bins and the corresponding first or second threshold emission signal level estimates.

In yet other implementations, the present disclosure contemplates a method for analyzing emission signal data from a digital PCR (dPCR) assay which includes generating a histogram from emission data collected from reaction sites of a dPCR assay, the histogram may include a plurality of bins representing a number of reaction sites at different levels of emission signal. The method also includes identifying a first subset of the plurality of bins, the first subset may include bins having low concentration based on a number of reaction sites per bin being less than or equal to a predetermined amount. The method also includes identifying a first bin in the first subset of bins that corresponds to a highest level of emission signal. The method also includes setting a first threshold estimate of emission signal level at the highest level of emission signal of the identified first bin. The method also includes identifying a second subset of the plurality of bins adjacent to and over a range of emission signal level lower than the first threshold estimate of emission signal level. The method also includes identifying a second bin in the second subset of bins that corresponds to a lowest level of emission signal. The method also includes setting a second threshold estimate of emission signal level at the lowest level of emission signal of the identified second bin. The method also includes comparing the population of data below the first threshold estimate to the population of data below the second threshold estimate. The method also includes based on the comparing, setting a threshold level for classification of the emission signal data as either the first threshold estimate or the second threshold estimate. The method also includes for a reaction site, classifying emission signal data from the reaction site as positive for amplification of target from the dPCR assay if the level of emission signal corresponding to the reaction site is above the threshold level or classifying the emission signal data as negative for amplification of target from the dPCR assay if the level of emission signal corresponding to the reaction site is below the threshold level.

The present disclosure further contemplates implementations including computer-readable media storing one or more instructions which, when executed by one or more processors of at least one computing device, cause the one or more processors to perform a method of any of the above claims. In yet another implementation, the present disclosure contemplates a system comprising one or more processors of at least one computing device, and a memory storing instruction which when executed by the one or more processors cause the one or more processors to perform a process in accordance with any of the methods above.

It is to be understood that both the foregoing general description and the following detailed description are exemplary and explanatory only and are not restrictive of the claims; rather the claims should be entitled to their full breadth of scope, including equivalents.

### BRIEF DESCRIPTION OF THE DRAWINGS

Various objects, features, characteristics, and/or advantages of embodiments of the present disclosure will become apparent and more readily appreciated from the following description of the embodiments, taken in conjunction with the accompanying drawings and the appended claims, all of which form a part of this application. In the Drawings, like reference numerals may be utilized to designate corresponding or similar parts in the various Figures, and the various elements depicted are not necessarily drawn to scale. In the Drawings:

FIG. 1 is an illustration of a scatterplot of dPCR assay data;

FIG. 2 is an illustration of a histogram depicting aspects relating to utilizing a distribution-based technique in determining an emission signal threshold for analysis of dPCR assay data;

FIG. 3 is an illustration of a histogram of data associated with measured emission signal from reaction volumes of a dPCR assay, with the x-axis representing emission signal level and the y-axis representing the count of reaction volumes emitting signal at the corresponding emission signal level;

FIGS. 4A and 4B are illustrations depicting aspects of one implementation of a technique for automated determination of a threshold emission signal level for use in calling reaction volumes from dPCR assay emission data;

FIG. 5 depicts a workflow in accordance with an implementation of the present disclosure for setting a threshold for use in dPCR assay data analysis;

FIG. 6 schematically illustrates a scatter plot and corresponding histograms of dPCR assay emission data to show the effect bin size can have on the determination of bins having low concentration (emptiness), and thus separation between clusters of data;

FIG. 7 shows a workflow illustrating how a first threshold estimate can be determined in accordance with an implementation of the present disclosure;

FIG. 8 shows a workflow illustrating how a second threshold estimate can be determined in accordance with an implementation of the present disclosure;

FIG. 9 shows a workflow illustrating how to set a final threshold estimate based for use in making positive and negative calls for reaction volumes of a dPCR assay;

FIGS. 10A and 10B are illustrations depicting aspects of another implementation of a technique for automated determination of a threshold emission signal level for use in calling reaction volumes from dPCR assay emission data;

FIG. 11 shows a workflow illustrating how a first threshold estimate can be determined in accordance with another implementation of the present disclosure;

FIG. 12 shows a workflow illustrating how a second threshold estimate can be determined in accordance with another implementation of the present disclosure;

FIGS. 13A-20 show example dPCR data sets for a variety of types of data and threshold determinations in accordance with implementations of the present disclosure;

FIGS. 21A-21B show data sets of double negative band with and without positive control data;

FIG. 22 shows an implementation of the use of control signal for threshold determination in accordance with the present disclosure;

FIG. 23 shows example data sets having dense poorly separated cluster and rainy data attributes and how threshold determination can be made using the techniques described herein and in FIG. 24; and

FIG. 24 shows example data sets and the effects of dynamic sub sampling and range adjustment.

### DETAILED DESCRIPTION

The present disclosure contemplates dPCR assay data analysis that provides a robust and automated technique for determining a threshold of emission signal level that is used to make calls of positive or negative for amplified product in a reaction volume.

Implementations of the threshold determination techniques provided herein do not rely on characterizing distribution of data based on data clusters and probabilities of a data point as being within a particular characterized distribution or not. More specifically, techniques for threshold determination in accordance with aspects of the disclosure eliminate the potential errors associated with distribution-based techniques in which a modality, such as normal, bimodal, or trimodal, of the distribution of the data analyzed is determined. These can be susceptible to error because such modalities can be difficult to discern for various types of data populations. Moreover, even when a modality is determined correctly, distribution-based techniques can pose challenges in differentiating between negative clusters of data that include more than one peak from positive clusters of data that may have a relative low peak.

To overcome shortcomings associated with distribution-based thresholding techniques, implementations of threshold determination techniques contemplated herein are separation-based and single population centric. More specifically, the threshold determination techniques in accordance with various implementations rely on finding separation of data by looking for relative “emptiness” (low concentration) of data adjacent a cluster of relatively high concentration. Instead of analyzing the measured data via characterizing a distribution of a cluster of high concentration data, techniques in accordance with various implementations of the disclosure involve organizing the data into a histogram, looking for bins in the histogram exhibiting relatively low concentration, and selecting thresholds that are determined as adjacent a cluster (single population) of high concentration data. The separation-based techniques in accordance with aspects of the disclosure can thus rely on identification of a cluster of data that is determined to be either positive or negative (single population of data) based on the emissions signal levels associated with the cluster, but the techniques do not rely on knowing if two clusters exist or assuming anything about the type of distributions represented in any cluster.

Moreover, to provide further robustness to the techniques outlined, control signals and various adaptive techniques can be implemented to determine attributes of the data sets presented and adjust parameters of the algorithms so as to improve accuracy in threshold determinations.

Techniques for threshold determination in accordance with various implementations of the disclosure can thus provide a relatively straightforward and mathematically simple technique to set a threshold automatically, thereby allowing for fast computation of the same for an array of dPCR data. For example, implementations in accordance with the present disclosure can output a threshold in 0.2 milliseconds (ms) to 20 milliseconds (ms) for 10000 to 20000 data points, such as from a dPCR assay using an array containing 10000-20000 reaction sites. For a range of from 100 to 1,000,0000 data points (reaction sites), the threshold can be output in 0.2 ms to 200 ms. Moreover, because techniques in accordance with various implementations of the disclosure can rely on a single cluster (population) of data and set the threshold based on the same, without having to rely on additional clusters, the time to determine a threshold can be reduced. A smaller range of the overall data can be utilized, with no need to make assumptions as to the modality of the overall data set.

Other advantages and distinguishing features of techniques for determining emission signal level thresholds for application to dPCR assay data in accordance with aspects of the present disclosure will be appreciated from the following discussion of various implementations.

The terms “determine,” “calculate,” and “estimate” are used synonymously herein. These terms are not intended to imply an exact level of measurement precision. Thus, where a value is “determined,” “calculated,” or “estimated” using the embodiments described herein, it will be understood that such a value may include some degree of inherent error due to factors such as detection instrument tolerances, rounding, chemical reaction variability, and other inherent measurement imperfections known and understood by those of skill in the art.

In addition, unless otherwise indicated, numbers expressing quantities, constituents, distances, or other measurements used in the specification and claims are to be understood as optionally being modified by the term “about” or its synonyms.

When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., substances, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

### Overview of Threshold Determination of Digital Polymerase Chain Reaction (dPCR) Assay Data

As discussed above, in biological sample analysis using dPCR assays, a large number of reaction volumes are subject to an amplification (e.g., PCR) reaction and reaction volumes that contain a target nucleic acid template produce an amplification product that produces a detectable emission signal by virtue of sequence-specific primers and/or probes (which carry a detectable label such as a fluorescent molecule) configured to specifically interact with the target nucleic acid of interest. Reaction volumes that do not contain the original target nucleic acid template do not produce the corresponding amplification product and thus do not emit an emission signal at a level indicative of the generation of such amplification product.

In such dPCR assays, a sample is diluted and segregated into the large number (several thousand) of reaction volumes, and emission (e.g., fluorescence) signal from the associated probe (fluorescent molecule) is detected from each of the large number of reaction volumes subject to the amplification reaction. The number of reaction volumes (or reaction sites of an array) can range several hundred to several million. For example, microwell plate arrays may be utilized in which generally multiples of 8, such as 96 or 384 wells, form the array of reaction sites. Other formats include so-called open arrays, such as arrays of through holes forming the reaction sites, Such open arrays may have several thousand reaction sites, such as from 3000 to 20000 reaction sites, for example from 10000 to 20000 sites. Some dPCR assays run multiple arrays at a time and thus can collect emission data from each of the arrays. In one application, emission signal can be collected from four open arrays at a time, with each array comprising 3000 reaction sites, but such is nonlimiting and exemplary only. Yet other reaction sites can be discrete reaction volumes (partitions) that are formed by drops or other discrete volumes of liquid in a flow or held statically in structures such as on slides or in conduits (e.g., capillaries). Such reaction volumes can be up to 5 million to 10 million, for example. The reaction volumes can be on the order of several picoliters to several microliters, depending on the format of the reaction sites. For example, reaction volumes of a microwell plate may range from 10 microliters to 20 microliters for example. Through hole reaction sites may have reaction volumes of about 400 picoliters, for example. Reaction volumes of discrete partitions or volumes of liquid in a flow or held statically can be on the order of 5 picoliters, for example.

In one representation of data collected from a dPCR assay, the data is organized as a scatter plot showing positional and/or consecutively numbered reaction volumes (e.g., contained at sites on a sample holder array) and the associated detected emission signal level (e.g., fluorescence intensity) corresponding to a particular wavelength (based on the wavelength of the detectable label used in the amplification reaction) from each reaction volume. By way of nonlimiting example, detection of emission signal can be detection of a signal within a particular wavelength range of fluorescence associated with the fluorescent molecule used as the detectable label in the amplification reaction. In various implementations, the emission signal measurement is fluorescence intensity quantified by a relative fluorescence unit (RFU), which is the amount of light within the wavelength band collected by the detector. Other implementations using other types of emission signals and mechanisms for detection of amplification, such as ions and the measurement of an electrical capacitance as the emission signal, are also contemplated within the scope of the disclosure and the use of fluorescent molecule labels and fluorescence emission detection is just one example and should not be considered as limiting. In other words, the present disclosure contemplates detection of other emitted electromagnetic signals for making calls in dPCR assays.

With reference to FIG. 1, an illustration of a scatterplot of dPCR assay data is shown. The data plotted is for emission signal data measured from over 20,000 reaction volumes subject to the dPCR amplification reaction, with each reaction volume assigned a unique number and plotted on the x-axis. The y-axis represents the measured emission signal level from each reaction volume, with the detection being associated with an emission wavelength of a detectable label (e.g., fluorescent molecule) used in the amplification reaction. A user selection of a threshold level of emission signal to base calls of positive and negative for amplification product in a reaction volume may be at the level associated with the line labeled T in FIG. 1, which represents an area between the two more densely populated clusters of relatively low emission signal level and relatively high emission signal level data labeled C_{L} and C_{H}, respectively. But due the data in the scatterplot between the clusters CL and CH being relatively “rainy” (sparsely and randomly positioned between the two clusters), determining at which emission signal level in the region between the CL and CH clusters the threshold T should be set makes an automated technique challenging. For example, the density in the region between C_{L} and C_{H}, while generally sparse, is substantially uniformly and randomly position, and thus challenging to determine where to put a threshold with a level of confidence based on determining density of data points.

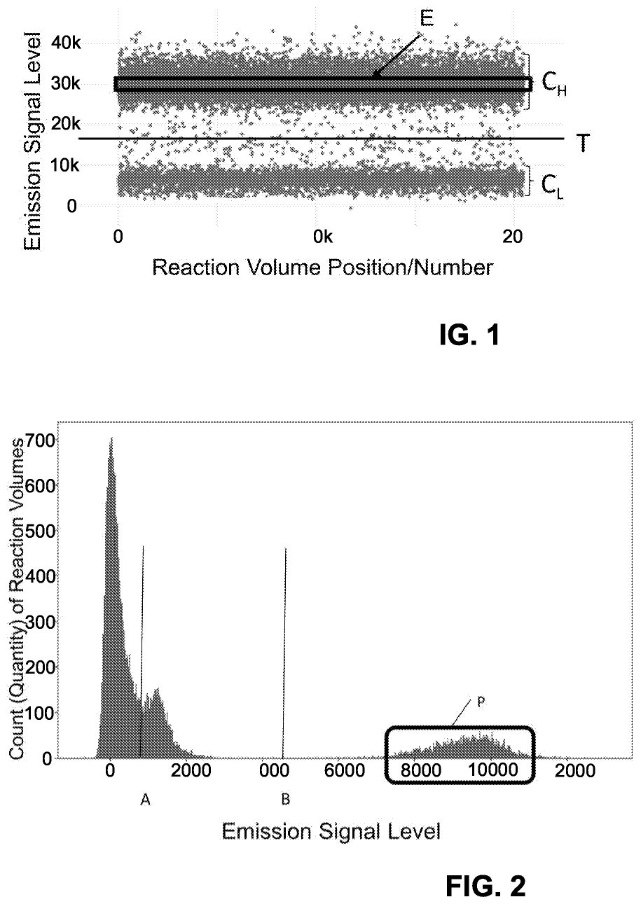

FIG. 2 is an illustration of some challenges associated with utilizing a distribution-based technique in determining an emission signal threshold for making positive and negative calls of dPCR assay data. A distribution-based technique can organize the emission data from a dPCR assay into a histogram, as shown in FIG. 2, with the x-axis indicating measured emission signal level and the y-axis indicating a count (quantity) of reaction volumes exhibiting that measured emission signal level. FIG. 2 illustrates how a data set may appear in a histogram from a dPCR assay in which a relatively large number of the reaction volumes are negative for the amplified product (and thus not containing the original target nucleic acid template or the amplified product thereof). FIG. 2 further illustrates how the data set may appear in the presence of a relatively low number of positive reaction volumes, labeled P on the plot.

In a data set in FIG. 2, with the existence of the positive cluster P of data points, and in view of the positive cluster P being relatively low count (peaks) and the double peak distribution of the negative reaction volumes with each of the peaks being higher than the peak of the positive cluster P, using a distribution-based model that assumes a bimodal distribution can result in an inaccurate threshold determination. For example, because of the nature of the data and its distribution, using a bimodal distribution assumption may erroneously set the threshold at A (i.e., between the two peaks of the negative cluster (low emission signal level) of data in FIG. 2) instead of correctly setting it at B (i.e., between the negative cluster and positive cluster P). In some cases, this challenge of using a distribution-based technique can be exacerbated because in some applications it may be untenable to know whether a sample will have any of the target (i.e., be positive) rather than none (i.e., entirely negative) before analysis.

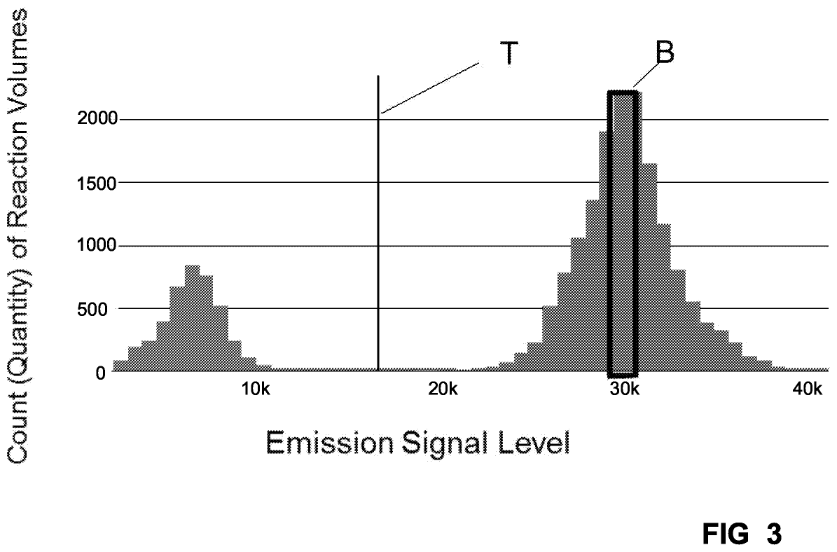

Threshold determination techniques in accordance with various implementations disclosed herein can address the challenges associated with the distribution-based or scatter plot user-based threshold determination techniques discussed above. With reference to FIG. 3, the data associated with measured emission signal from reaction volumes of a dPCR assay can be organized as a histogram with the x-axis representing emission signal level (e.g., fluorescence intensity) for the emission wavelength associated with the detectable label (e.g., fluorescent molecule) used in the amplification reaction and the y-axis representing the count (quantity)of reaction volumes emitting signal at the corresponding emission signal level. With reference again to FIG. 1, the correlation between data as represented in a scatterplot format and the histogram format of FIG. 3 can be seen. More specifically, each bin of the histogram (one such bin B being depicted) maps to an emission signal range in FIG. 1 (one such range E being depicted). The corresponding threshold T from FIG. 1 is also illustrated as T in FIG. 3.

As can be seen from FIG. 3, organizing the data in the histogram format illustrated and looking for “emptiness” adjacent a cluster of data and corresponding separation between clusters of data to the extent more than one cluster exists, rather than distribution within the clusters themselves, can provide a robust and efficient technique for determining a threshold. Such a technique can be more reliable than relying on distributions that may be difficult to determine the mode of distribution or to accurately place thresholds in the presence of some types of data, such as has been explained above with reference to FIG. 2. A further explanation of various implementations of techniques relying on finding such emptiness adjacent a cluster of histogram data will now be discussed in more detail.

### Threshold Determination Technique Based on Identifying Low Concentration Bins Adjacent a High Concentration, Single Population (Negative or Positive) Cluster

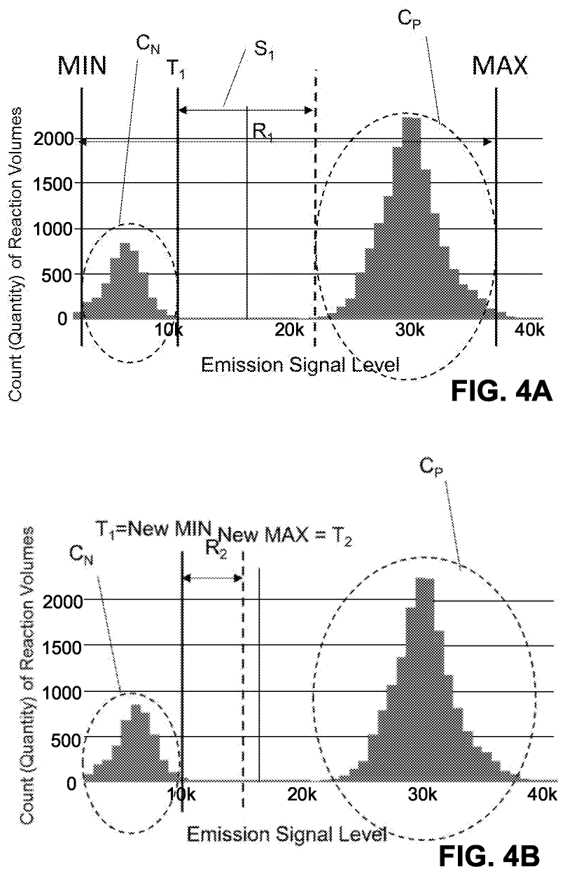

With reference to FIGS. 4A and 4B, a schematic illustration of an implementation of a technique for automated determination of a threshold emission signal level for use in calling dPCR emission data is depicted. As discussed above with reference to FIG. 3, the measured emission signal data (which may include fluorescence intensity in a specified wavelength associated with a fluorescent molecule detectable label for example) may be organized as a histogram of bins of the number of reaction volumes (y-axis) for a given emission signal level range (x-axis). A minimum and maximum of emission signal level within the overall data set can be set (reflected by the “MIN” and “MAX” labels in FIG. 4A) so as to ensure outlier data is excluded from the threshold determination analysis.

After setting the minimum (MIN) and maximum (MAX), the data falling between that range can be utilized to guess an initial threshold, labeled T₁ in FIG. 4A. The initial threshold guess at T₁ in FIG. 4A is determined by identifying a first subset of bins, within the plurality of bins between a search range R₁ defined between the minimum (MIN) and maximum (MAX) emission signal levels. The identified bins of the first subset S₁ are those having low concentration (in terms of the count/quantity of reaction volumes in each of the bins). After defining the search range R₁ and first subset of bins S₁, the bin of the first subset of bins having the lowest level of emission signal is identified. This corresponds to where the negative population cluster C_{N} ends in a direction along the positive x-axis. Stated differently, the first threshold guess T₁ is the emission signal level adjacent to a cluster of bins in an emission signal level range associated with negative amplification, labeled C_{N} in FIGS. 4A and 4B.

In various implementations, the identification of the subset of bins S₁ that is considered low concentration and thus as “empty” for practical purposes, can be determined by calculating a low concentration (i.e., quantity of reaction volumes/bin) threshold value and identifying bins in the range R₁ that are below such concentration threshold value. For example, the low concentration threshold value can be calculated as a sufficiently low percentile of the total number of data points plotted in the histogram, such as for example, a percentile ranging from 0.05% to 10%, such as, for example 0.1% of the total number of data points between the MIN and the MAX.

Once the first threshold estimate T₁ is determined, a second threshold estimate T₂ is determined by setting a new search range R₂ of bins (based on a newly defined minimum (New MIN) and maximum (New MAX)). The second threshold estimate T₂ is determined by finding the highest emission signal level within the second subset of bins corresponding to the search range R₂. The second subset of bins and new search range is intended to encompass a search range above (i.e., beginning at higher emission signal levels) than the first search range between the initial minimum (MIN) and maximum (MAX) values.

Reference is made to FIG. 4B, which schematically depicts the determination of the second threshold T₂. A new search range R₂ is identified by using the first threshold estimate T₁ as the new minimum (New MIN). A new maximum (New MAX) is set and can be based on various calculations, as described further below. The objective in setting the new maximum (New MAX) of the search range R₂ is to ensure the defined threshold is set sufficiently above the negative cluster C_{N} but below the beginning of any cluster of data representing data points associated with emission signal levels indicating reaction volumes that are positive for amplification product. FIG. 4B schematically depicts such a cluster as the group of data with relatively high concentration toward the higher emission signal levels in the scatter plot and labeled as C_{P}, indicating a cluster of data corresponding to positive reaction volumes.

FIG. 4B illustrates an exemplary new maximum (labeled New MAX) and the second subset of bins making up the new search range R₂ of the plurality of low concentration bins. Within this new search range R₂, the highest emission signal level is identified and set as a second threshold estimate T₂, which is equal to the highest emission signal level of a bin of the new search range R₂. In the illustration of FIG. 4B, T₂ coincides with the new maximum (New MAX), but this is nonlimiting and T₂ could be another bin and emission signal level within the search range not necessarily equal to the new maximum.

Notably, in an entirely negative population of data points (i.e., no positive cluster C_{P}), T₁ could equal the maximum MAX of the entire first search range. Accordingly, the new search range could exceed the first, with the new maximum (New MAX) exceeding the initial maximum.

The process of narrowing the search range can be an iterative one based on various criteria, examples of which are described further below with respect to various implementations. Finding the first threshold estimate in accordance with various implementations provides a threshold outside of, even though adjacent to, a cluster of data for which there is a degree of confidence that the cluster represents negative or positive (a single population) for amplification product. This can in turn provide confidence that the threshold will be in a region of separation between two such clusters if both populations exist and can be utilized as a threshold. For example, in the scenario depicted in FIGS. 4A and 4B, the first threshold estimate T₁ is chosen as adjacent to the cluster of data CN representing reaction volumes that emit signal associated with being negative for amplification product. Implementations described further below may instead provide a first threshold estimate as adjacent to a positive cluster of data.

The second threshold estimate attempts to find a threshold that is further away from the first threshold estimate in the region of separation between clusters of negative and positive data if both populations exist, but without running into the cluster of data that the first threshold estimate was not adjacent to (e.g., the positive cluster of data CP in the scenario illustrated in FIGS. 4A and 4B). As can be seen in FIG. 4B, the second threshold T₂ is further within the low concentration bin range (region of separation) between the negative and positive clusters of data C_{N}, C_{P}, allowing for more assurance that the threshold is in a sufficient area of separation between the two to provide more reliable calls. Generating the new search range and set of bins within that range and above T₁ also can allow for a finer check to optimize the threshold placement and ensure the final placement of the threshold is in a low concentration set of the data (bins).

To make a final threshold determination with which to compare against the emission signal data measured for each reaction volume so as to call the reaction volume as positive or negative for amplification product, the standard deviation of the population of data points below the first threshold estimate T₁ is compared to the standard deviation of the population of data points below the second threshold estimate T₂. If the two calculated standard deviations are relatively close, with the standard deviation of the population below T₂ being slightly higher, then T₂ can be set as the final threshold for use in the data analysis for making positive and negative amplification product calls.

If however, the standard deviation of the population below the second threshold estimate T₂ is significantly higher than the calculated standard deviation below the first threshold estimate T₁, then it is an indication that T₂ has been set to an emission signal level within or exceeding the positive population. In this case, T₁ can instead be set as the final threshold for use in the dPCR data analysis for making positive and negative amplification product calls. Alternatively, one or more iterations of altering the search range and subset of bins can be used and the highest emission signal level within the new search range can be identified as the second threshold estimate and again compared to the calculated standard deviation as above. Utilizing the above-described comparisons of standard deviations of the populations below T₁ and below T₂ represents one technique to determine the threshold. However, this is nonlimiting and various other metrics, such as, but not limited to, for example, total emission signal range of each population, averaging the signal range of each population, counts of data points in each population, or some other composite variable to evaluate the shapes of the population below T₁ and T₂, respectively, and confirm they are not significantly different may be used. Stated differently, the analysis attempts to ensure T₂ captures a small portion of events not captured by T₁, but is not so far away from T₁ so as to capture a positive population and cause it to be classified as negative. A composite value may include a sum, product, or ratio of other metrics, such as, for example, taking a difference of the means above and below the threshold and dividing them by the sum of the standard deviations above and below the threshold, or any other mechanism for quantifying/scoring the clustering created by the thresholds.

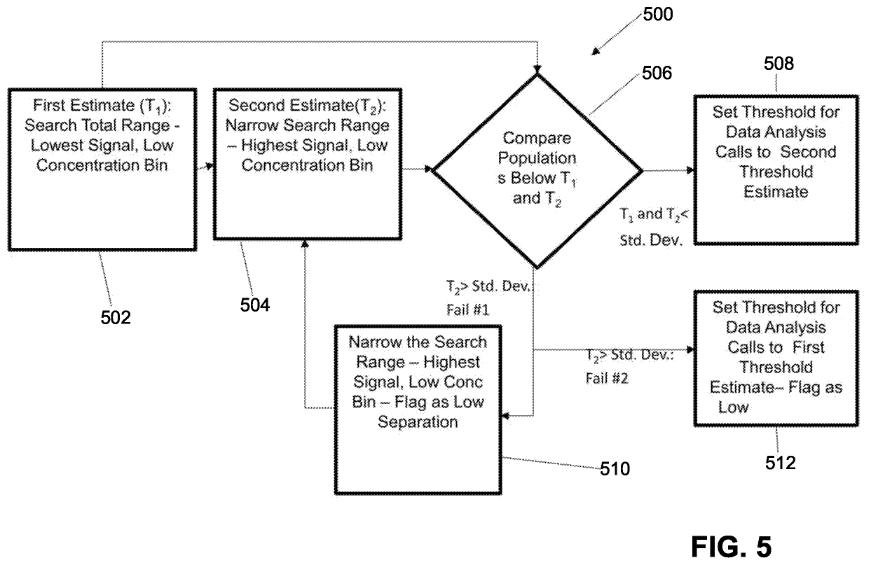

FIG. 5 depicts one implementation of a workflow in accordance with aspects of the present disclosure for setting a threshold for use in dPCR assay data analysis for making a determination (call) as to whether a reaction volume is positive or negative for amplification product based on comparison of its measured emission signal to the threshold.

In the workflow 500 of FIG. 5, at action 502, a first total range of data is searched. The total range of data searched can cover emission signal levels ranging from 0 to the highest emission signal level measured, or alternatively can cover a range from an initial minimum emission signal level to a maximum emission signal level (e.g., between the MIN and MAX emission signal levels depicted in FIG. 4A). Setting the initial range between a different MIN and MAX than zero and the highest measured emission signal level can remove outlier data. Within that first total search range at action 502, a first subset of bins of the histogram can be identified as low concentration bins and corresponding emission signal level range (S₁ as depicted in FIG. 4A). As described above, the identification of low concentration bins can be made by setting a low concentration threshold value as described above with reference to FIGS. 4A-4B. Once the subset of low concentration bins S₁ in the range R₁ is identified, the lowest emission signal level within that subset of bins can be identified and set as the first threshold estimate (e.g., T₁ in FIG. 4A). This sets the first threshold estimate at an emission signal level that is in the low concentration subset of bins and adjacent (just to the right of) the negative cluster of bins (e.g., C_{N} in FIGS. 4A and 4B).

Next, at action 504, the search range of bins can be narrowed from the initial search range R₁. Narrowing the search range can be accomplished in a variety of ways, such as using the first threshold estimate as the minimum of a new search range and setting the maximum of the new search range as a factor based on the population of either the negative population of data or the expected positive population, or some other search criteria, including a random new maximum for the search range. Once the new search range is identified at step 504, which will encompass the low concentration subset of bins in the search range again identified, the highest emission signal level within the new range of low concentration bins is identified and set as a second threshold estimate (e.g., T₂ in FIG. 4B).

At action 506, populations below both the first threshold estimate and the second threshold estimate can be compared (e.g., such as the standard deviations of the emission signal levels or other metric as described above). If the comparison indicates the populations are comparable (allowing for the population beneath T₂ being slightly larger than that below T₁, then the workflow 500 proceeds to action 508 and the final threshold for use in the dPCR data analysis for making positive and negative calls for reaction volumes is set to the second threshold estimate T₂.

If, however, the population below the second threshold estimate T₂ differs significantly from the population below the first threshold T₁, then the workflow 500 proceeds to action 510 and the search range is narrowed again. For example, the previous search range used for the second threshold determination can be narrowed by a factor, such as ranging between ¼ to ¾ of the search range, or for example, 1/2 the search range, starting the range at T₁ as the new minimum for the next search range The new, narrower search range can be used and actions 504 and 506 repeated again with the new search range. If after the new search range is used and the new second threshold estimate set based on that search range, the population below the second threshold estimate T₂ differs significantly from the population below T₁, then the workflow 500 proceeds to action 512, with the final threshold for dPCR data analysis calls being set to the first threshold estimate. Optionally, at action 512, the threshold can be flagged as “Low” or some other indicator and provided as feedback, so that it may prompt a user to consider a manual adjustment to the threshold if desired.

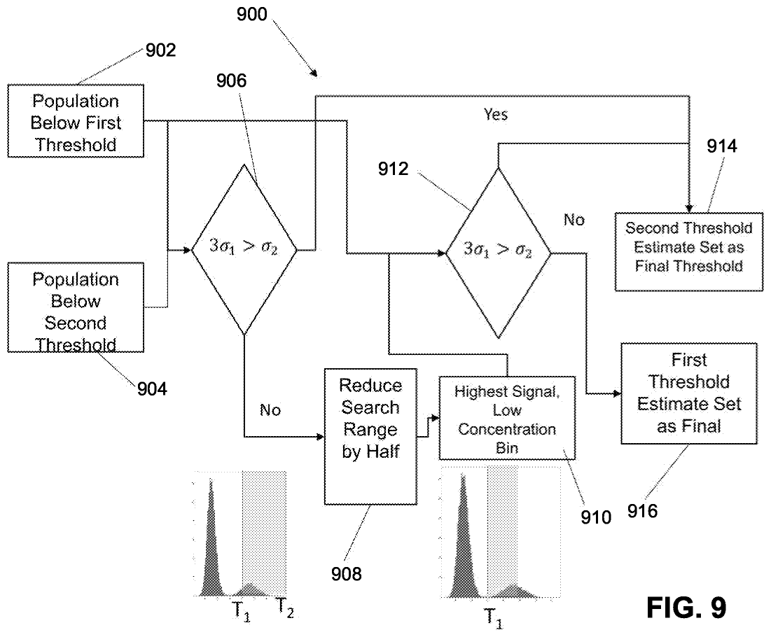

As noted above, the comparison of the populations below T₁ and T₂, respectively, in the workflow 500 can be based on the use of various metrics to characterize the populations. In one implementation, described with reference to FIG. 9 below, the standard deviations of the two population is used to determine if the standard deviation of the population below T₂ is significantly higher (e.g., more than two times, for example more than three times) than the standard deviation of the population below T₁ so as to determine whether to narrow the search range and/or determine which of the first threshold or second threshold estimate to use as the final threshold emission signal level for dPCR data analysis.

It should be appreciated that while the workflow 500 utilizes two iterations of narrowing the search range and setting two second threshold estimates for the comparison at action 506, additional narrowing and iterations could be performed as desired. Performing the additional iterations may increase the overall processing time, however, for setting the threshold, and may not lead to a significant improvement in accuracy of calls by using the first threshold estimate after the search ranges have been narrowed in two iterations.

As can be understood from the above description of implementations for determining a threshold using the histogram and emptiness adjacent single population techniques described above, initial inputs for finding the low concentration bins can include determining the minimum and maximum range of the emission signal levels to search (if it is desired to attempt to throw out outlier data) and the bin size (i.e., the range of emission signal levels encompassed by each bin of the histogram). The bin size that is selected affects the determination of the existence of sufficient emptiness and separation between negative and positive clusters (populations) of data. Stated differently, the bin size can affect whether a bin can be discerned as low concentration (or “empty” for practical purposes).

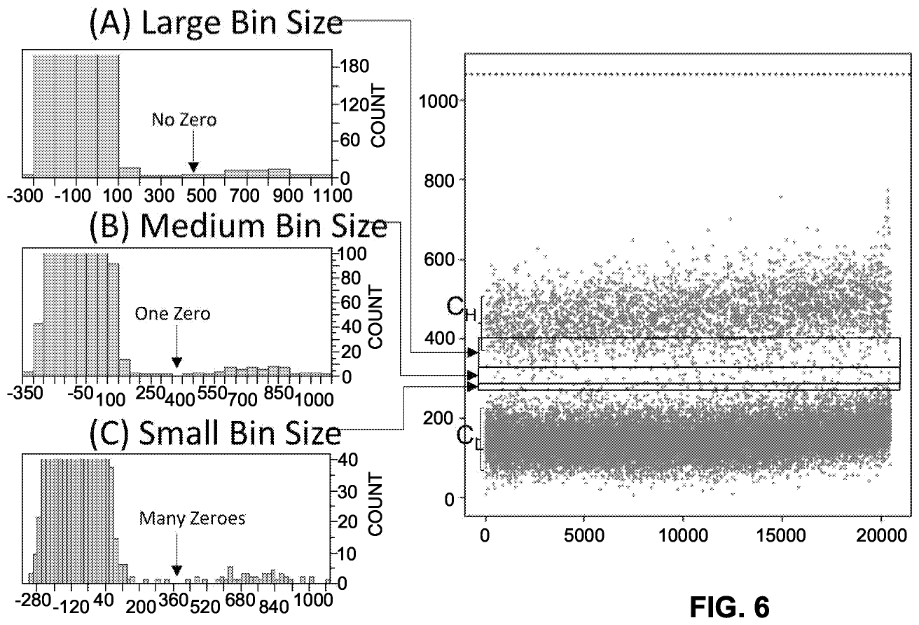

FIG. 6 schematically illustrates the effect that bin size can have on the determination of bins having low concentration (emptiness), and thus separation between clusters of data. The right hand portion of FIG. 6 is a schematic illustration of a scatter plot of dPCR data, similar to the scatter plot representation of FIG. 1. In the scatterplot shown, the data collected is indicative of a sample having a large number of reaction volumes that are negative for amplification product. Utilizing the data as observed in the scatter plot of FIG. 6, a generally sparser cluster of data can be seen between the relatively low and high clusters (labeled CL and CH respectively as in FIG. 1). Within that sparser cluster of data, three different bin sizes are illustrated. The depicted bins are not necessarily to scale but rather are illustrative of the concepts described with regard to how bin size can affect the number of data points captured in a bin. The left hand portion of FIG. 6 schematically illustrates the representation of the data in the scatter plot as a histogram, similar to the histogram representation of FIG. 3. Each of the three histograms A, B, and C in FIG. 6 respectively corresponds to using the differing bin sizes (relatively large, medium, and small sized bins) represented in the scatter plot of FIG. 6.

As can be seen, the bin size can affect how many empty (as reflected by the number of bins showing as empty in the histograms) bins are presented, with smaller bins sizes having more and larger bins sizes less (and potentially none). In an implementation, bin size can be selected such that the algorithm for the auto-thresholding can select a threshold for similar data sets and such that such selection generally agree with human interpretation of the data. For example, if CH in FIG. 6 should be considered as a negative population of data points, a relatively large bin size can be selected. If the CH population is on the borderline of acceptability as a positive population of data points, a medium bin size can be selected. If CH is to be categorized as a positive population of data points, a relatively small bin size can be selected.

The bin size can be determined in various ways. In accordance with one implementation, the bin size can be set based on the expected emission signal level corresponding to a reaction volume positive for amplification product. The expected signal level associated with a positive call can be determined by running a control for positive as part of the dPCR assay and determining the mean emission signal from such control reaction volumes or can be predetermined based on a prior dPCR assay with the same reaction parameters. The bin size can be selected based on setting a nominal percentage of the expected positive emission signal level. In an implementation, the nominal percentage can range from 0.1% to 10%, for example, from 0.2 % to 5 %. The nominal percentage can be decided based on the performance of the positive control of the assay. Poorly separated data may indicate selection of a smaller bin size while higher signal data may benefit from the use of a larger bin size.

Thus, each bin can represent a uniform emission signal level range that is equal to the nominal percentage of the expected positive emission signal level.

As discussed above, in addition to setting the bin size, the initial maximum and minimum of the emission signal level can be set to determine the overall range of data that will be used for the initial threshold estimate.

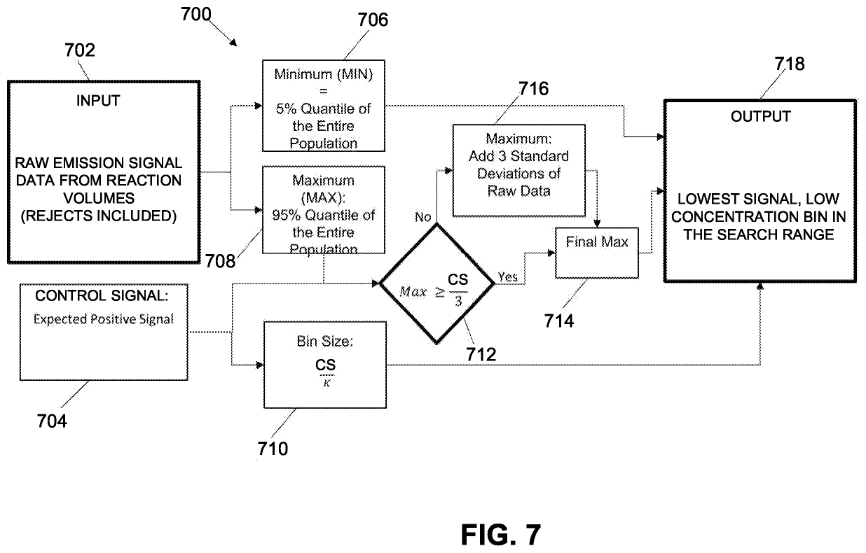

Referring now to FIG. 7, a workflow according to an implementation in accordance with aspects of the present disclosure is illustrated. The workflow 700 shows the various inputs and actions according to one implementation for setting the search range (based on setting the initial maximum and minimum of emission signal levels as described with reference to FIGS. 4A and 4B for example) and bin size to proceed with the determination of the first threshold estimate.

The workflow 700 begins with inputs at actions 702 and 704 respectively. Specifically, at 702, the raw emission signal data from the reaction volumes of a dPCR assay is provided and organized as a histogram with any rejects of data excluded.

Rejects of data can be based on a number of factors and can include unreadable data and/or significant deviations from expected emission signal level ranges. Further input at 704 can include a control signal that corresponds to the emission signal level associated with an expected positive emission signal level, as discussed above. The control signal can be determined by the system based on a control used as part of the dPCR assay, with the mean emission signal levels from the control being used to set the global variable. In other implementations, the control signal can be predetermined based on prior dPCR assays and set in auto threshold software program, or it can be input by a user.

Based on the inputs at 702 and 704, the workflow 700 determines minimum (MIN) and maximum (MAX) emission signal levels at actions 706, 708 so as to define the initial range of data to be used in the search for the first threshold estimate. In the workflow 700, the minimum emission signal level can be set as the 5% quantile of the entire population of the data set provided by the input at 702. The maximum emission signal level can be set as the 95% quantile of the entire population of the data set provided at 702. These ranges are chosen so that 90% of the data can be included in the initial search range. But this can be adjusted and the 5% and 95% quantiles for setting the minimum and maximum emission signal levels are nonlimiting and exemplary and can be altered to encompass other ranges of the data. For example, the defined range for the minimum level can range from 0% to 40 %, or for example, from 0% to 20% quantile of the population of emission data. The maximum emission signal level can range from 60% to 100%, for example, from 80% to 100%, or from 90% to 100%. Thus, the initial search range can be defined at the emission signal levels between the set minimum and maximum emission signal levels determined at 706, 708, respectively.

From the input provided at 704, the workflow 700 sets a bin size used for the threshold determination at action 710. The bin size can be set as the expected positive signal from the control signal provided as input at action 704 divided by a number that can correlate to small enough bin sizes to capture the anticipated expected positive signal. As noted above, the bin size should ultimately reflect the minimum amount of separation needed to categorize a cluster as positive or negative. For control positive data that is poorly separated, a smaller bin size can ensure the data is still classified as having positive data points. If, on the other hand, chemical/spectral artifacts are anticipated which may affect some data points appear to separate from the negative cluster when they are not actually positive, a larger bin size can result in additional robustness to avoid classifying these artifacts as positive.

In the implementation of FIG. 7, the control signal (CS) is divided by the number of bins, which can be selected based on using a fraction of the expected positive emission signal level. In various exemplary embodiments, to account for a relatively large expected positive emission signal level, the number of bins can be based on a percentage ranging from about 0.1% to about 10% of the expected positive emission signal level, such as, for example, from 0.2% to about 5%. For example, for an expected positive emission signal level of 5000 RFU, using approximately 250 bins, the bin size would be about 20 RFU per bin, which represents about 0.4% of the expected positive emission signal level of 5000 RFU. The number of bins can be determined experimentally and/or preset given a particular type of instrument and assay being run.

While the implementation of FIG. 7 can rely on a percentage of the expected positive signal to determine bin size, the bin size can be determined in other ways without departing from the scope of the present disclosure. For example, Sturges' Formula can be employed and the following relationship used: K=1+3.322*log(N) where N is the total number of datapoints and K is the number of equal size bins to be used across the data and be entered into the #Bins at action 710.

In various implementations, the bin size may also be automatically determined based on the type of data presented, and machine learning could be used to recognize patterns of the presented data and adjust the bin size accordingly. For example, bin size can be adjusted to provide more tolerance for “rainy” poorly separated raw data. Once approaching a small enough bin size, e.g., <2% of the expected positive emission signal level, bin size may be optimized based on how tolerant the algorithm should be to “rainy” poorly separated data, with smaller bins (e.g., on the order of 0.1% of the expected positive emission signal level) resulting in more tolerance to bad signal separation, and larger bins (e.g., on the order of 1% of the expected positive emission signal level) resulting in less tolerance.

At action 712 of the workflow, the control signal CS is utilized to determine if the maximum emission signal level determined at action 708 is viable for setting the first range by which to determine the first threshold estimate. For example, as shown, CS can be divided by 3 and the maximum value MAX determined at action 708 can be compared to that quantity. If the maximum value determined at 708 is greater than or equal to the quantity CS/3, then the maximum from 708 can be used as the final maximum value at 714 and used to set the maximum limit of the range of emission signal levels to be used in the search range for the determination of the first threshold estimate at action 718 (e.g., using the technique disclosed with reference to FIG. 4A and action 502 of FIG. 5 as described above.)

If at 712, the maximum value MAX determined at 708 is not greater than or equal to CS/3, the workflow 700 proceeds to action 716 and the maximum can be recalculated by adding 3 standard deviations of the raw emission signal level data from the input at 702 and setting the maximum to that quantity to be used as the final maximum MAX at 714 and provided into the determination of the first threshold estimate at action 718.

The ratio of CS represents a fraction of the total expected signal and is chosen to represent a soft threshold by which a data set can be predicted to be mostly negative, but allowing some positives in the dataset to exist. As used herein, a soft threshold can refer use of control signal, including a fraction of data relating to the control signal, to coarsely characterize a data set. Aside from this fractional, mathematically straightforward approach, this could also be accomplished by estimating the modality/number of peaks within the dataset as well.

The workflow 700 can thus conclude by using the determined minimum (MIN) and maximum (MAX) values to set the range of data to be included to determine the low concentration bins, as described above. Then from that determination, the first threshold estimate can be determined based on the identification of the lowest emission signal level of identified low concentration bins, as described above with reference to FIG. 4A and action 502 of FIG. 5 for example.

Utilizing the lowest emission signal level of a low concentration bin as the first threshold estimate T₁, in accordance with various implementations described above, ensures that the first threshold estimate will be adjacent (touching) the negative population of data points (e.g., cluster C_{N} of FIGS. 4A and 4B). Increasing this threshold estimate (e.g., using the second threshold estimate T₂) assumes that this first threshold estimate is mostly correct in its ability to separate negative calls from positive calls.

Assuming a fairly wide separation between the two clusters and/or a sparse concentration of data over the low concentration bins, raising the threshold to the second threshold estimate level is not expected to affect the standard deviation of the population beneath the threshold.

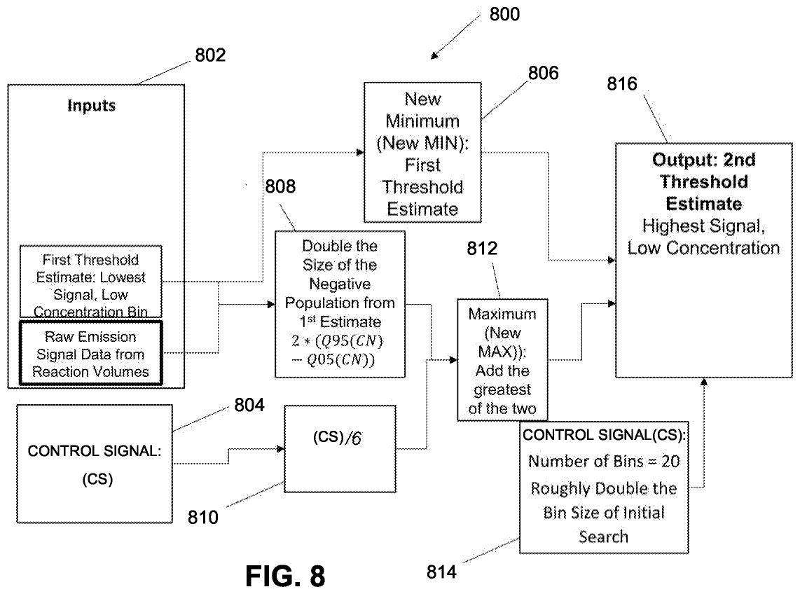

Given the assumptions above regarding the relative accuracy of the determination of the first threshold estimate, FIG. 8 shows a workflow illustrating how the second threshold estimate can be determined. The workflow 800 of FIG. 8 begins with inputs at actions 802, 804. Action 802 represents the input from the output 718 of FIG. 7, namely the first threshold estimate and the raw emission signal data from the dPCR assay reaction volumes. As part of the output of 718, and thus forming the input of action 802, the initial minimum (MIN) and maximum (MAX) defining the initial search range and the bin size also are included. The input provided at action 804 includes the control signal CS, determined as discussed above with respect to action 704 of FIG. 7.

As discussed above with reference to FIGS. 4B and 5, to determine the second threshold estimate, the initial search range is adjusted (narrowed). At 806, the new minimum (New MIN) for the search range is set as the first threshold estimate. To determine the maximum emission signal level (New MAX) value for the new search range, at action 808, the size of the negative population can be doubled from that used in first threshold estimate determination. For example, the value at 808 can be calculated from 2*(Q95(C_{N})-QO5(C_{N})), where Q95(C_{N}) and Q 05(C_{N}) represent the 95th and 5^{th} quantiles of the population of the negative cluster CN (i.e., data below T₁).

At action 810, the control signal CS can be divided by 6 based on taking a portion of the expected positive emission signal level. Once the calculations at 808 and 810 are made, at action 812, the greater of the two values calculated at 808 and 810 can be used as the new maximum (New MAX) defining the adjusted search range. The goal of actions 808-810 is to increase the search range by an amount that does not exceed the positive population C_{P} because surpassing it would result in the definition of highest signal, low concentration bin (for the second threshold estimate T₂) as above the positive population C_{P} of data points.

Using the criteria ⅙ of the expected positive emission signal and two times the size of the negative population as noted above can provide a mathematically straightforward way to raise the threshold to an appropriate level, but is one nonlimiting implementation. Other ways to accomplish this can include, for example, testing for a minimum number (e.g., 5) of consecutively low bins (as binned by steps outlined in 700) and selecting the highest bin of the consecutive bins as T₂ (which can entirely forgo determining the highest signal, low concentration bin approach) or binning a range between T₁ and CS using a relatively large bin size (5-10 bins) and selecting the lowest signal low concentration bin.

Alternate ways to setting a new maximum and finding the highest signal of a low concentration bin can include testing a number of bins (as binned from steps outlined in 700) (e.g., 20 to 50 additional bins) and selecting the highest signal of a low concentration bin; finding the maximum by fitting a distribution curve to the cluster beneath T1, and placing the new maximum where the fitted curve intercepts a sufficiently low density (e.g., 0.1-0.001); or setting the maximum manually based on human interpretation of positive control data.

With the new search range defined between the first threshold estimate T₁ as the minimum (New MINS) set at action 806 and the new maximum (New MAX) as determined at action 812, the second threshold estimate T₂ can be determined by finding the highest signal, low concentration bin in the new range of bins at action 816. In addition to determining the new minimum and maximum to define the new search range, the bin size can optionally also be adjusted in the workflow 800 for determining the second threshold estimate. As reflected at action 814, the bin size based on the control signal determined from the first threshold estimate workflow (e.g., action 710 of workflow 700 in FIG. 7), as described above, can be doubled and used as the new bin size in the determination of the second threshold estimate at action 816; alternatively, Sturge's rule can be utilized.

The second threshold estimate determined at action 816 can be utilized and recalculated based on a new search range using the workflow 500 of FIG. 5 or otherwise as described above. A workflow for determining whether to utilize the initially calculated second threshold estimate or to narrow the search range and recalculate the second threshold estimate is illustrated in FIG. 9. In workflow 900 of FIG. 9, the population of data points below the first threshold estimate (e.g., as calculated using the workflow of FIG. 7) is used as an input at action 902 and the population of data points below the second threshold estimate (e.g., as calculated using the workflow in FIG. 8) is used as an input at action 904. At action 906 a multiple of the standard deviation of the population below the first threshold estimate (σ₁) is compared to the standard deviation of the population below the second threshold estimate (σ₂). For example, as illustrated in workflow 900, 3*σ₁ is compared to σ₂. If 3*σ₁ is found to be greater than 02, the second threshold estimate determined from the workflow 800 is used. If it is not, the search range determined for use in the second threshold estimate is reduced again (for example by ½) at action 908 and a new second threshold estimate determined at action 910 as the highest emission signal level of the low concentration bins in the new search range.

After action 910, the workflow 900 moves to action 912, where again the first threshold estimate from action 902 and the updated second threshold estimate determined at action 910 after narrowing the search range are utilized as the inputs for determining the standard deviations σ₁ and σ₂. Notably, σ₁ does not change between actions 906 and 912, but σ₂ does because the second threshold estimate has changed and thus the population below it and the standard deviation of that population also have changed. At action 912, a comparison of 3*σ₁ and the new σ₂ is made and if 3*σ₁ is greater than the new σ₂, the new second threshold estimate is set as the final threshold to be used for the dPCR assay data analysis for making positive and negative calls for amplification product in the reaction volumes at action 914; otherwise, the first threshold estimate is set as the final threshold at action 916. Qualitatively, the comparison is seeking to capture a cutoff point at which the positive population has been captured below the second threshold estimate T₂ based on finding that too many events, and thus too large of a signal disparity, have been categorized as negative as compared to those events captured below the first threshold estimate T₁.

The histogram plot at the left in FIG. 9 represents what the raw data and first and second threshold estimates (labeled T₁ and T₂, respectively) may look like in the scenario where the second threshold estimate is too high and thus the relationship at action 906 is not satisfied. To address the situation, the search range can be reduced, for example by ½, as reflected in the histogram plot at the right in FIG. 9. Because the new, narrower search range in that histogram ends within the second, positive cluster of data, the highest emission signal level of a low concentration bin should be found adjacent to the left side of that cluster (within the range indicated) and thus can be used as the final threshold. If halving the range still had the maximum past the positive cluster of data toward the right, however, the relationship at action 912 would again not be satisfied and the first threshold T₁ would be used as the final threshold.

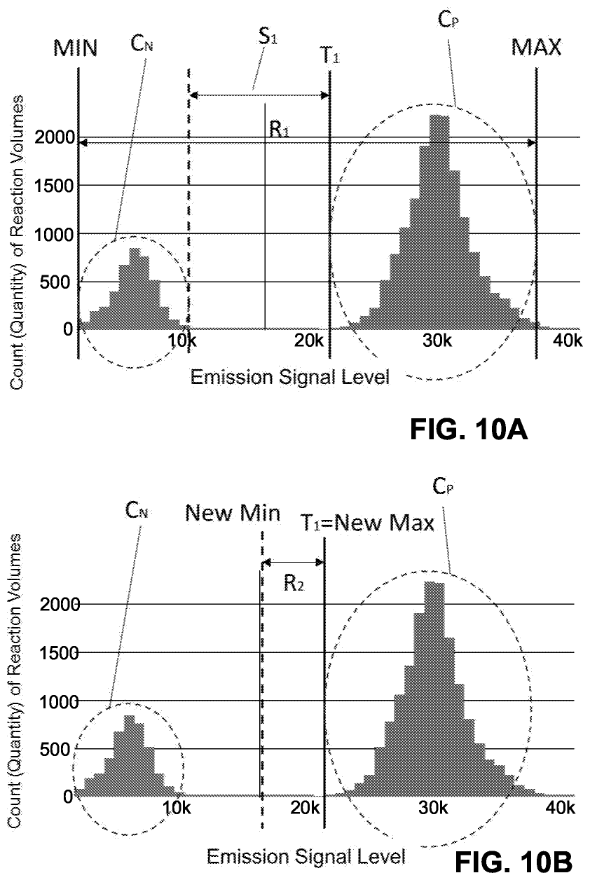

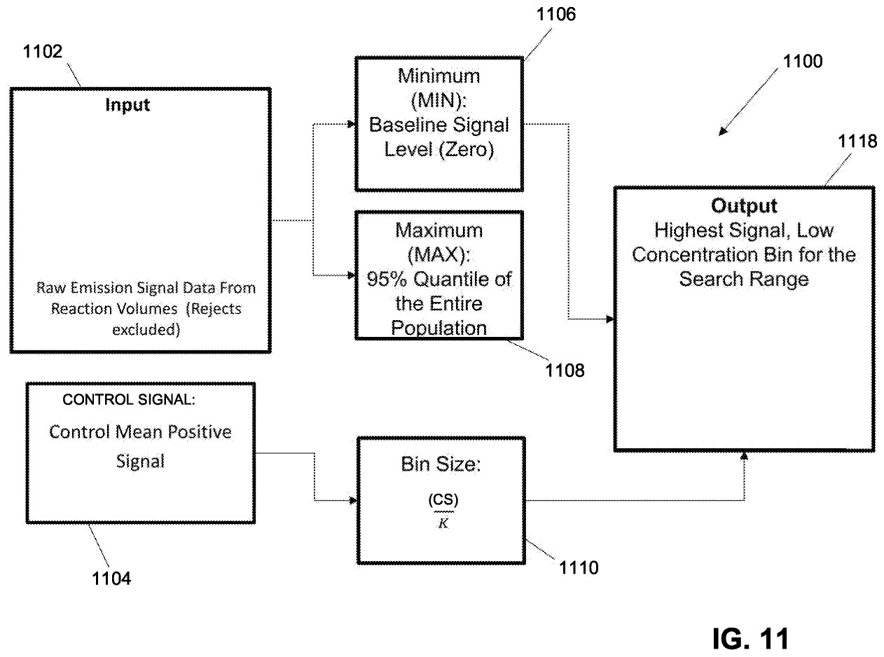

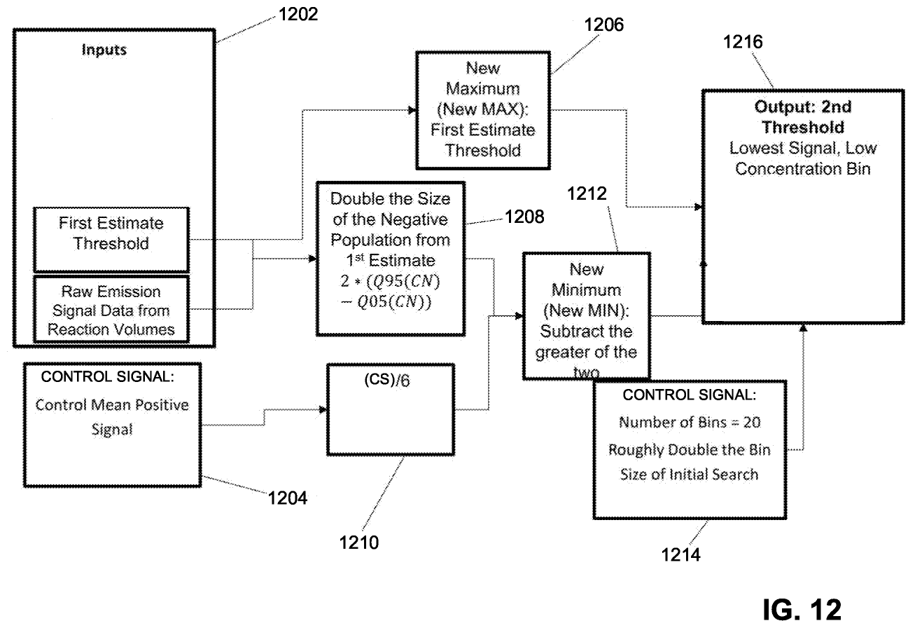

While various of the implementations described above utilized the negative population (cluster) (e.g., C_{N} in FIGS. 4A and 4B) and the “empty” bin in an emission signal range above and adjacent to that cluster to set the first threshold estimate, and then to narrow the ranges from there and use the highest emission signal level bin for the second threshold estimates, the present disclosure also contemplates implementations that utilize the positive population (cluster) of data as the basis for the thresholding estimates based on the separation (emptiness) techniques herein. That is, with reference to FIGS. 10A and 10B, the positive cluster of data C_{P}) can be identified, a maximum (MAX) and minimum (MIN) set to define a search range R₁, and the low concentration bins determined (S₁) based on some relatively low percentage of the concentrations associated with the positive cluster of data. Then, the “empty” bin in an emission level range below and adjacent the positive cluster C_{P} of data (highest emission signal level in low concentration bin range) can be identified as the first threshold estimate T₁. To determine the second threshold estimate T₂ (e.g., moving farther way from T₁ toward lower emission signal levels but with the objective of not moving into the negative cluster of data C_{N}, the search range of bins (emission signal level search range) can be narrowed in a manner opposite to that described with reference to FIGS. 4A and 4B. That is, T₁ can be set as the new maximum (New MAX) and a new minimum for the search range R₂ can be found. The implementation of using the positive cluster of data can be understood based on the principles described above with respect to using the negative cluster of data as the starting cluster for determining the first and second threshold estimate and example workflows are illustrated in FIGS. 11 and 12 for identifying the first and second threshold estimates T₁ and T₂, final threshold, the minimum and maximum for the initial search range, and the new minimum and new maximum for the narrowed search range The workflows 1100 and 1200 of FIGS. 11 and 12 are similar to those described above with reference to FIGS. 7 and 8 except working from the positive cluster and toward the negative, with various actions of the workflows 1100 and 1200 of FIGS. 11 and 12 having reference labels similar to those of FIGS. 7 and 8, respectively, except for the first digits being 11XX and 12XX instead of 7XX and 8XX, respectively.

The workflows 1100 and 1200 are described briefly here, with other aspects discussed with reference to the workflows 700 and 800 of FIGS. 7 and 8 not specifically discussed for the sake of simplicity, but may apply as would be understood by those having ordinary skill in the art based on the disclosure herein. The actions 1102, 1104, 1108, and 1110 are the same as actions 702, 704, 708, and 710 discussed with reference to FIG. 7. At action 1106, the initial minimum (MIN) for the initial search range can be the baseline signal level and thus encompass essentially zero emission signal level.

The minimum (MIN) determined at 1106, the maximum (MAX) determined at 1108 and the bin size determined at 1110 are input at action 1118 and used to set the range of data to be included to determine the low concentration bins, as described above. Once the low concentration bins are identified, the first threshold estimate of emission signal level is set by identifying the highest emission signal level of the identified low concentration bins and that first threshold estimate is the output of the workflow 1100.

The workflow 1200 can be utilized to find the second threshold estimate of emission signal level when starting from the positive cluster C_{P}. Actions 1202, 1204, 1208, 1210, and 1214 are the same as actions 802, 804, 808, 810, and 814. At action 1206, a new maximum (New MAX) for a new search range is set to the first threshold estimate determined at 1118 in the workflow 1100 of FIG. 11. At action 1212 a new minimum (New MIN) for a new search range is found by subtracting the greater of the values determined at action 1208 and 1210. The New MIN and New MAX, and optionally a new bin size from action 1214, are input at action 1216. The new search range between the new minimum emission signal level (New MIN) and the new maximum (New MAX) is searched and the lowest emission signal level in the bins of the new search range is identified and set to the second threshold estimate at action 1216. Other aspects for determining whether to set the final threshold estimate to the first or second threshold estimates can be implemented in a manner similar to that described above with reference to FIG. 9.

Various considerations can factor into a decision as to whether to use a negative population of data points (e.g., C_{N}) or a positive population of data points (e.g., C_{P}) to find the threshold estimates. One such consideration is the expected positive signal. Comparing the mean of the total raw data against the expected positive signal can provide one technique for determining which of the negative and positive clusters of data is the starting cluster for the thresholding implementations described herein. If the mean signal is greater than some factor (such as ½) of the expected positive signal, then the data can be assumed to be predominantly positive and the positive cluster CP can be used as the starting cluster (e.g., following the implementations of FIGS. 10A-12). Otherwise, the negative cluster CN can be the starting cluster (e.g., following the implementations of FIGS. 4A, 4B and 7-9).

Another technique for determining which cluster is the starting cluster for the thresholding implementations is to compare a count of how many data points exceed a predetermined soft threshold. For example, a soft threshold can be set as a factor (such as ⅓ to ½) of the expected positive signal and the data points exceeding that can be counted. If a sufficiently high count of the total data points is positive (e.g., greater than 50%-80%, then the positive cluster Cp can be used as the starting cluster ((e.g., following the implementations of FIGS. 10A-12). Otherwise, the negative cluster CN can be the starting cluster (e.g., following the implementations of FIGS. 4A, 4B and 7-9).

Other techniques to making the determination that do not utilized the expected positive signal include machine learning based flags (e.g., setting either flags for positive-like data or negative-like data) of individual reaction volume data and then counting whether the majority of the flags are set to positive or negative to make a decision as to whether there are more likely positive or likely negative data. This technique may not be as robust to multiband and/or rainier sets of data, and may not align with human perception of whether something should be positive or negative.

Yet another technique can be based on a distribution or weight-based estimation of the data. For example, if the total raw data appears to be “top heavy” (positive-centric) based on simple distribution assessments (e.g., skewness of distribution or whether mean exceeds median, etc.), then the positive cluster can be selected as the starting cluster for the thresholding. A drawback of this approach may be that overly negative data points due to spectral crosstalk or other external factors may create an overly negative signal and thus negatively impact the ability of this approach to discern whether the data is positive or negative centric.

Overall, the principles of relying on an “empty” bin adjacent a single cluster (population), which further correlates to separation between two clusters if both exist, apply regardless of whether the cluster being used is for a negative or positive population of data. An implementation relying on the positive cluster of data as the starting point for thresholding can be useful for data sets that are saturated with few or no negatives being presented.

While various implementations described above set the initial MIN and MAX values of the emission signal levels to be used for the initial search range at 5% quantile and 95% quantile of the entire population of data, such is nonlimiting and other ranges are contemplated. For example, the minimum (MIN) emission signal level can be in a range of 0% quantile to 40% quantile of the entire population of data, or from 0% to 20%, and the maximum (MAX) emission signal level can be in a range of 60% to 100%, for example, from 80% to 100%, for example from 90% quantile to 100% quantile of the entire population of data.

The determination of the maximum is intended to ensure that even in an entirely negative dataset, the maximum possible threshold estimate still captures about 90% of the entire negative dataset. Using a lower quantile for the maximum (e.g., 80%) may still be viable but will sacrifice low concentration/zero concentration performance and may require broader search ranges for additional threshold guesses. Similarly, raising the minimum can result in increased tolerance to outliers but may result in poor performance with predominantly amplified data, such as, for example if less 20% of all events are negative.

### Example dPCR Assay Data and Threshold Placement

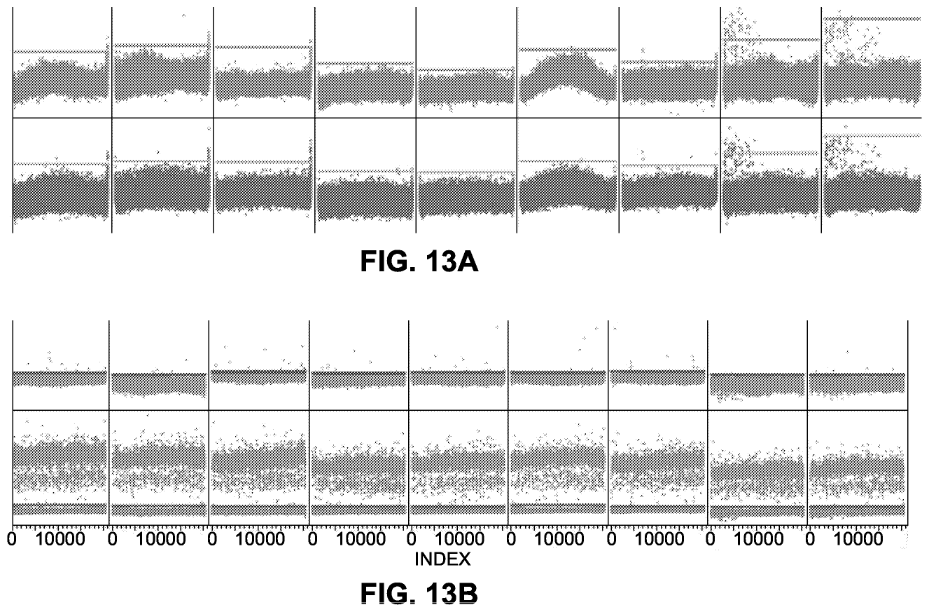

Example dPCR assay data and placement of thresholds for the same utilizing the emptiness-based, single population (cluster) centric thresholding techniques in accordance with various implementations described above are provided in FIGS. 13A-20. The figures show scatter plots of data with the index number of the reaction volume from an array subjected to a dPCR assay on the x-axis and the measured emission signal (fluorescence intensity of label/dye of interest) on the Y-axis. The exact values are not important to show the implications of the data and therefore in instances where those numbers are not presented or are difficult to read, it is not essential to understanding what the data is conveying as described below. The different data shown in FIGS. 13A-20 are generally labeled by the type/result of data shown and conclusions of what the data demonstrates for each set of data represented are summarized below.

FIGS. 13A and 13B show example dPCR data sets with placement of a first threshold estimate T₁ based on the determination of the lowest signal, low concentration bin adjacent the negative population, as described for example, with reference to FIGS. 4A-9 and the corresponding written description above. FIG. 13A highlights two datasets in a multiplex reaction which have been determined to be entirely negative based on human interpretation, this highlights that, while T₁ does not place the threshold sufficiently above all datapoints in these negative datasets, it sufficiently captures the majority of all data. FIG. 13B highlights an example of a dataset containing both negative (top) and positive (bottom) datasets which demonstrate acceptable accuracy in the placement of T₁. When positives are present (bottom), they accurately discriminate the positive cluster from the negative cluster, but in a predominantly negative dataset (top), higher threshold placement would be desirable to eliminate the low signal datapoints that do not fit into the main negative cluster. These datasets reinforce the assumption that T₁ captures the majority (>90%) of all negative datapoints in a dataset beneath it. Raising the threshold to above the first threshold estimate can occur under the assumption that this first threshold estimate has accurately classified the majority of the negative population, which assumption can be verified if the raised threshold does not significantly affect the standard deviation from the raised threshold of the population beneath the raised threshold as compared to that of the standard deviation from the first threshold estimate of the population beneath the first threshold estimate.

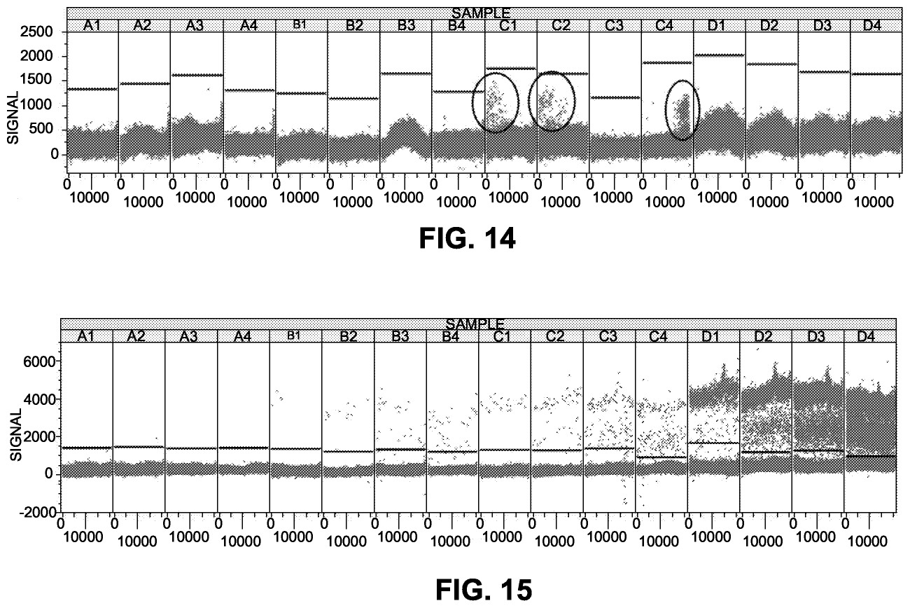

FIG. 14 shows example dPCR data sets in which the negative data includes baseline abnormalities and the thresholds (solid, black, horizontal lines) determined using the techniques described above. The three data sets that are circled represent data that from a human observation shows data not randomly distributed across the array of the reaction volumes. Moreover, the circled data for the data sets is also significantly dimmer (lower in intensity) than expected for a positive reaction volume. Accordingly, from a human perspective, a threshold is desired that would lead to the circled data being classified as negative. The solid black, horizontal line in each of the data sets represents a threshold determined by the auto-thresholding techniques (emptiness-based, single population (cluster) centric thresholding) of described above. The data and threshold line indicate that the thresholding techniques in accordance with the present disclosure is correctly determined to ensure the baseline negative abnormalities are below the threshold.

FIG. 15 show example dPCR data sets with negative data and increasingly high concentration of positive data (concentration of positive data increasing from left to right in the data sets), as well as the thresholds (solid, black, horizontal lines) determined using the techniques described above. As can be observed in the data sets, as the sample concentration increases, the positive signal becomes poorly separated (i.e., the data in the positive signal becomes poorly separated from the negative. As can be seen from the placement of the threshold line, however, the techniques above allow the threshold to be placed lower because the placement is based on finding the lowest concentration bins. In addition, the multistep iterative process allows for high placement of the threshold in low concentration/negative data, and low placement of the threshold in poorly separated data. This can ensure that minimal false positives are called from negative data and, vice versa that minimal false negatives are called from positive data.

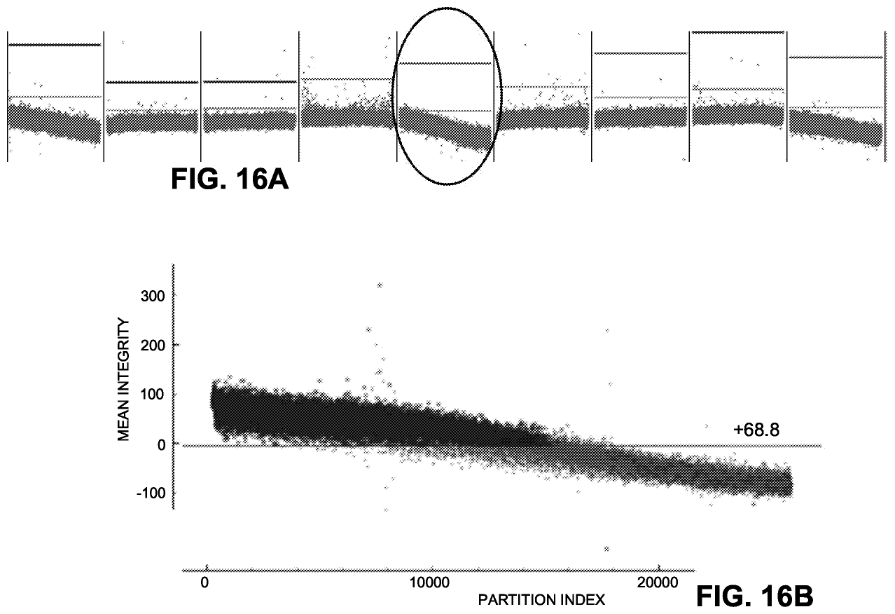

FIG. 16A shows example dPCR data sets with slanted baselines and the thresholds (T₁ shown in solid, horizontal, lighter lines and T₂ shown in solid, horizontal, darker black lines)determined using the techniques described above. FIG. 16B shows a detailed view of the circled data set in the middle of the various data sets of FIG. 16a and threshold placement (horizontal line) based on a technique other than those described herein. As shown by the placement of the threshold lines in FIG. 16A, using the thresholding techniques in accordance with the present disclosure and as described above, the threshold is not placed in the middle of a population as the techniques will look for a lower concentration bin. In contrast, thresholding techniques not utilizing the techniques described herein and using other distribution based approaches could place the threshold in the middle of the population, leading to calling negative data as positives (represented by the dots above the line in FIG. 16B.

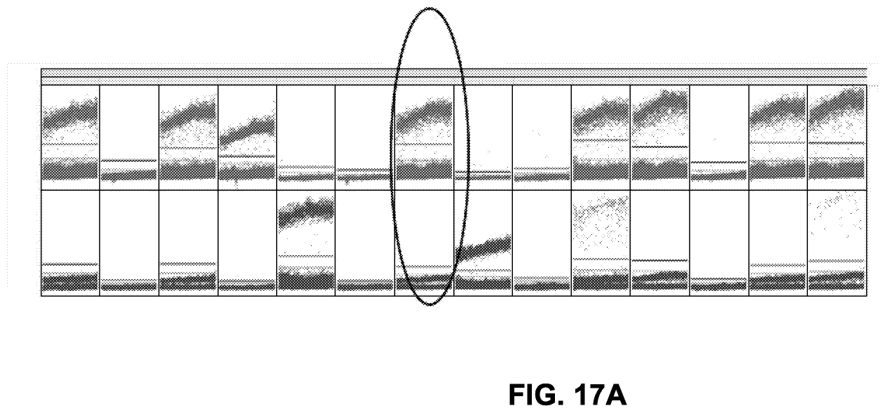

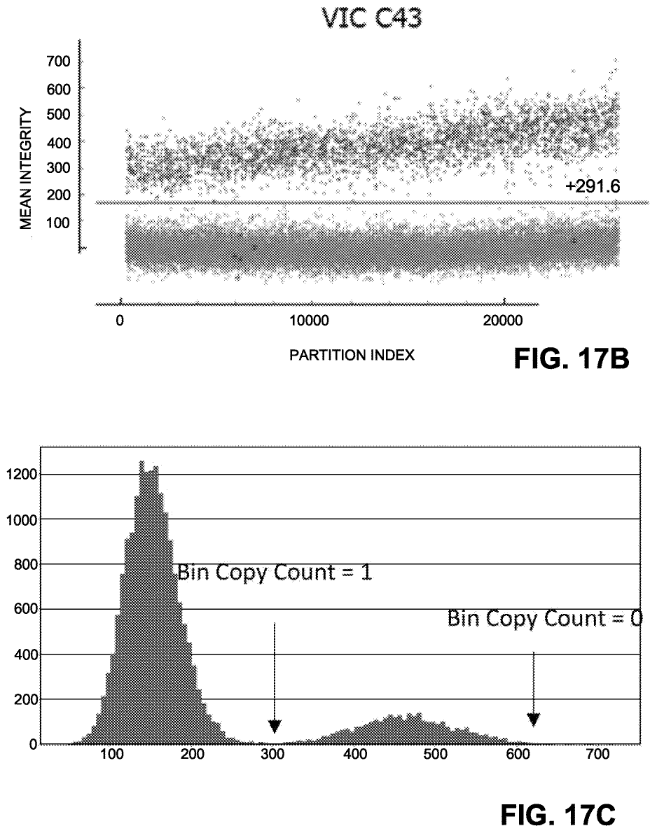

FIG. 17A shows example dPCR data sets generated by a genotyping multiplex reaction with strong negative double bands of data, as well as the thresholds (solid, black, horizontal lines) determined using the techniques described above. The top row of datasets was generated from one optical filter, and the bottom row of datasets was generated by a second optical filter. Assays are expected to generate signal only in one filters, but this data highlights a challenge wherein the amplification of assay in the top filter results in signal both in the first optical filter, but also generates a false-positive secondary band in the second optical filter. FIG. 17B highlights the dataset circle in FIG. 17A when processed using a classical auto-thresholding approach, without the context of a positive control a conventional approach will attempt to place a threshold between the two clear clusters in this dataset. FIG. 17C demonstrates the histogram view of the methods used to find the thresholds placed in FIG. 17A. histogram plot of the data set circled in FIG. 17A. The data in these figures demonstrate the thresholding technique described above in accordance with the present disclosure is able to ignore double negative bands. In other words, the determined threshold does not incorrectly separate the double negative bands into positive data calls and negative (split the double negative bands). Even without the use of a positive control sample (such as in Sample B1, B4, or C2) to help inform the thresholding and discern between negative and positive data (the use of which is described further below) based on intensity, the techniques described above can utilize expected signal level to correctly threshold and distinguish/classify positive populations from negative ones, thereby correctly choosing a threshold that includes both negative bands below the threshold, as reflected in the bottom set of data sets of FIG. 17A. This ability is dependent on the relationship of bin size with expected/control signal. An accurate control signal ensures that the minimum bin size is large enough to reject these double negative bands.

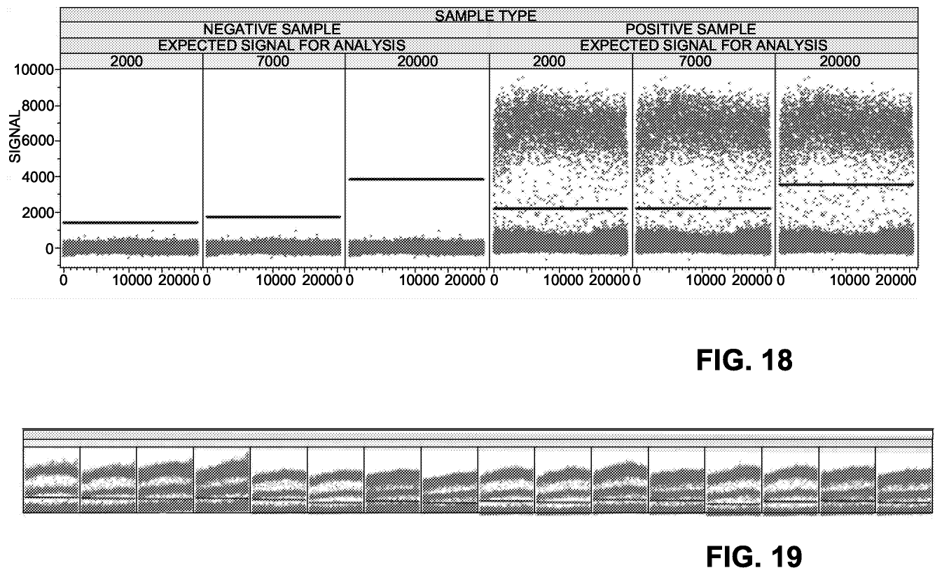

FIG. 18 shows example dPCR data sets, as well as the thresholds (solid, black, horizontal lines) determined using the techniques described above based on the expected signal indicative of positive sample being chosen inaccurately. The three data sets on the left are negative only sample data in which the expected positive signal values were selected as 2000, 7000, and 20000, respectively (as reflected at the top of the data sets). The three data sets on the right are for a sample with positive data and the same expected positive signal values of 2000, 7000, and 20000, respectively.

However, as can be seen by the data sets on the right, the actual positive values are in a range above 4000. As can be seen in the negative data sets, the threshold determinations accurately call all the negative data as negative (below the threshold) regardless of the expected signal value. For the positive data sets on the right, the threshold techniques are still able to produce acceptable thresholds regardless of the accuracy of the positive expected signal value, illustrating that using the methodology of finding the lowest concentration bins performs robustly. It is worth noting, however, that inputting a relatively accurate expected positive signal value when data is more challenging for desired results. Using positive control data, as discussed further below, can therefore be useful to provide accuracy in the expected positive signal when implementing the thresholding techniques described herein, in particular for challenging data sets.

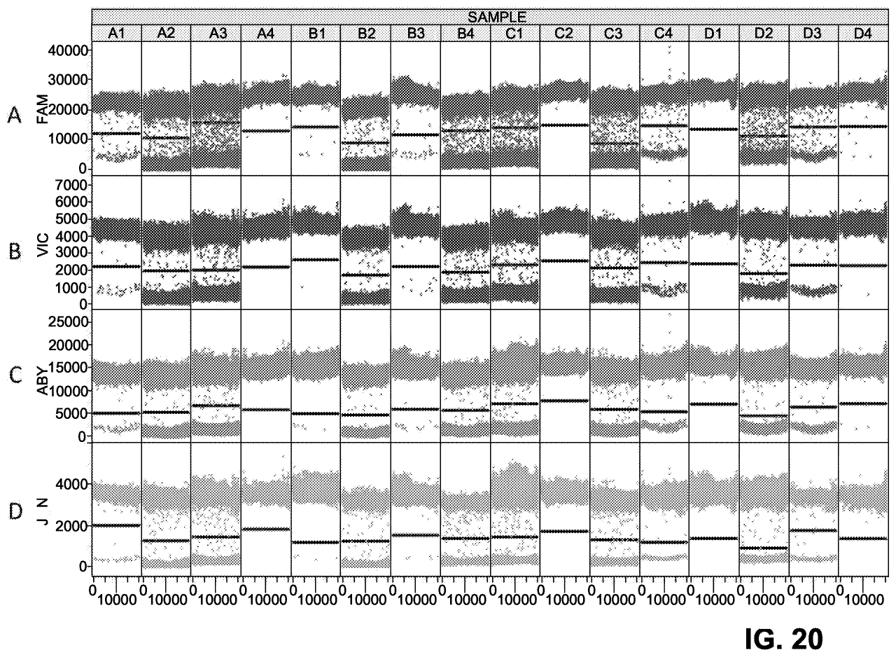

FIG. 19 shows example dPCR data sets, as well as the thresholds (solid, black, horizontal lines) determined using the techniques described above. This dataset represents a reaction in which multiple assays which have the same fluorophore amplify in the same optical channel. These will generate more than one positive cluster, in this example the middle cluster represents reactions where only one of the two assays generates positive signal in a microchamber. The top-most cluster represents reactions where both assays have amplified in the same microchamber, resulting in a greater combined signal level. FIG. 19 highlights that the thresholding techniques described above are still able to accurately discriminate both positive clusters from the single negative cluster.

FIG. 20 shows example dPCR data sets that include saturated data in which there are no negatives in the generated data, as well as the thresholds (solid, black, horizontal lines) determined using the techniques described above. The data sets illustrated correspond to sixteen samples (A1-A4, B1-B4, C1-C4, D1-D4) at or near saturation in a multiplex configurations utilizing four different fluorescent dye labels (FAM, VIC, ABY, and JUN) for the dPCR assays. Within each data set for a dye (i.e., the respective rows labeled FAM, VIC, ABY, and JUN, some of the data sets include saturated data with no negatives occurring, reflecting that each reaction volume of the assay for that sample was positive for amplification corresponding to that dye/label.

The data demonstrates that the thresholding techniques in accordance with those described above work even in the absence of negative data and appropriate thresholds can be determined using the positive population (cluster) as the base starting point, for example as described with reference to FIGS. 10A-12 and the corresponding written description thereof.

### Threshold Techniques Using Control Signal

As discussed above, the thresholding techniques in accordance with the present disclosure based on single population (negative or positive) clusters and emptiness (e.g., low concentration bins) adjacent such clusters have robust application in accurately determining thresholds for use in making positive and negative calls for reaction volumes in various types of data that may be obtained from dPCR assays.

Such data sets including, for example, normal populations with differing amounts of positives (e.g., as shown in FIG. 15), data sets that are difficult to group (e.g., as shown in the lowest set of data in FIG. 13B); data sets with a high concentration of positive compared to negative (extreme positive count as shown, e.g., in certain of the saturated data sets of FIG. 20); data sets with a double negative band (e.g., as shown in FIG. 17A, various of the bottom row of data sets) or a double positive band (e.g., as shown in FIG. 19, row A); and data sets with baseline abnormalities (e.g., as shown in FIG. 14).

Other data sets may present more challenges for implementing the thresholding techniques described herein and in such cases, use of a control signal may be desirable. Moreover, even for the data sets above that can provide relative accuracy in the determined thresholds, use of a control signal may nevertheless help improve robustness and/or efficiency in the threshold determination. The control signals described below can be used as the expected mean positive signal (control signal CS) when implementing the thresholding techniques described above with reference to the various workflows illustrated and discussed in FIGS. 7, 8, 11, and 12, for example.

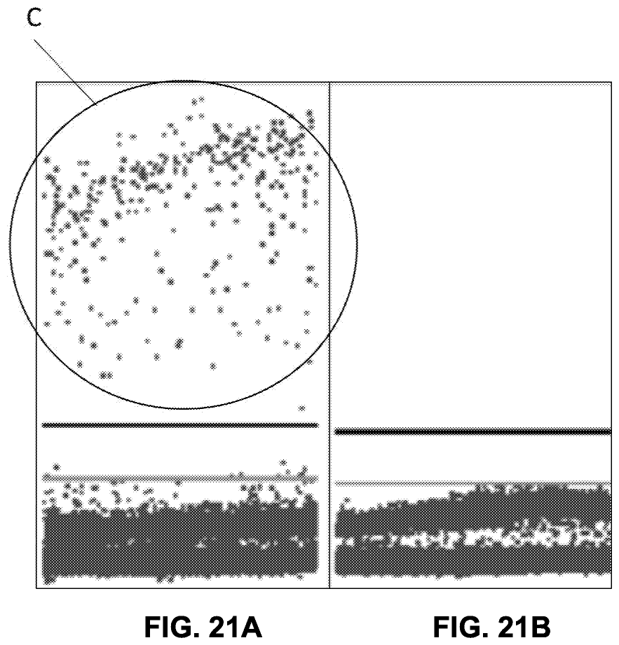

In various implementations, therefore, a control signal can be used to aid in informing the thresholding algorithm to make sense of clusters than can be difficult to comprehend in a vacuum. For example, where a single cluster exists it may be all positive or all negative, but determining that without a control signal may be challenging. Moreover, with reference to FIGS. 21A and 21B, in data sets with double bands (shown in FIG. 21B, it can be difficult for the thresholding algorithm to determine if two relatively distinct clusters are both negative (or positive) and in without additional information, the thresholder may determine the threshold to be placed between the two cluster, such that one cluster would be called positive and the other negative. Use of a control signal, as reflected by the data C in FIG. 21A, however, can provide further context and information regarding signal strength and thus allow the more accurate determination of the threshold to call both bands negative in the case of FIG. 21A. Overall, the use of a control signal in the thresholding techniques described herein can inform the algorithm (auto-thresholder) signals that are indicative of “real” amplification of the target. In other words, signals that correspond to a true positive for a reaction volume.

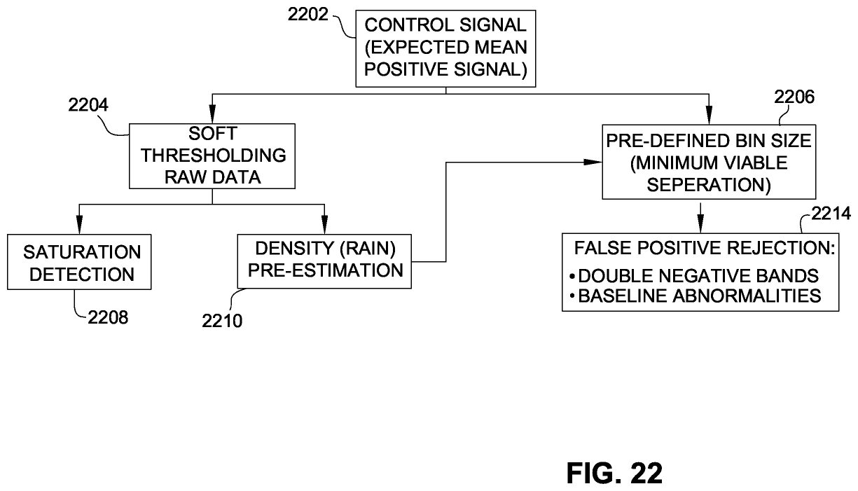

FIG. 22 illustrates how a control signal can be used in various parts of a workflow for implementing the thresholding techniques described herein. As shown, the control signal at 2202 can be used as the expected mean positive signal (control signal, CS, in the work flows described above). Knowledge of the control signal, and thus the expected mean positive signal, can be used to determine soft thresholds of the raw data as shown at 2204 and described further below and/or can be used to inform the pre-defined bin size for use in the thresholding techniques as shown at 2206 for use in the above workflows by informing a minimum viable separation. In turn, the soft threshold at 2204 can be used for saturation detection at 2208 and/or for making a pre-estimate of density of the data at 2210 so as to make a determination if the dataset is “rainy.” The density pre-estimation at 2210 can in turn also be fed into the determination of a pre-defined bin size to be used in the thresholding determination. By helping to inform a bin size, the control signal thus can assist in threshold selection so as to achieve false positive rejections at 2214, such as from sources such as double negative bands and baseline abnormalities as highlighted above, thereby improving the thresholding determinations.

Control signals can be determined in a variety of ways, including both deriving the control signal intrinsically from the dataset itself or extrinsically from a separately run dPCR assay with the control sample, for example, a positive sample that produces amplicons in the reaction volumes. For an intrinsic determination, the control signal can be determined algorithmically using a classifier (e.g., using artificial intelligence/machine learning algorithms and/or probabilistic algorithms), from a first-pass auto-threshold determination, and/or manual inspection of the data by a user and setting of the positive control signal threshold based on the same. For an extrinsic determination, the control signal can be determined by the mean signal of a positive control data set, this is only possible in a scenario where the assays are always being analyzed with the presence of a positive control. Instead of performing a positive control with every experiment, a general control signal can still be derived by characterizing an assay's performance with a positive control broadly across equipment, operators, and reagents prior to analysis. It is envisioned that these various approaches could also be used together, for example, a default control signal determined extrinsically could be set and used for the thresholding techniques, with an override allowed by a user based on intrinsic determination from the dataset.

Control signal can be used and implemented to serve a variety of purposes for threshold determinations in accordance with the techniques described herein. For example, as discussed above with reference to FIG. 22, the control signal at 2202 can be used to initially set a soft threshold (2204 in FIG. 22). For example, a soft threshold could initially be set equal to about ⅓ of the control signal, which can allow for an estimate of the total number of positives to determine if a sample (based on the emission signal obtained from the reaction volumes) is predominantly negative or predominantly positive. Setting the soft initial threshold to ⅓ of the control signal should capture approximately the highest signal (about 95% quantile) of the negative cluster in a sample (data set) that is about 50/50 positive and negative signals. If the amount of data captured is not the 95% quantile of the negative data, then this can be used to make the determination that the data is predominantly negative or positive.

In another implementation, a soft threshold can be set equal to about ⅖ of the control signal to estimate whether negatives exist at all in a predominantly positive data set. Selecting a soft threshold at this level can capture the lowest signal (about 5% quantile) of the positive cluster of an entirely positive sample. If significant negative data is captured, then it can be determined that the data is not predominantly positive, As also discussed with reference to FIG. 22, the control signal at 2202 can be used to inform bin size at 2206, such as the minimum bin size used to identify the separation between a positive and negative cluster (population) of data, as described in the various workflows and FIGS. 4-12 above. For example, the bin size can be set to 1/250 of the control signal, as described with reference to FIG. 7 for example, which in a data set with both positive and negative clusters can be used to provide a bin size representing roughly 0.4% of the control signal. Those having ordinary skill in the art would recognize that this may depend on the number of data points and the overall signal and be adjusted accordingly.

Yet another use for the control signal can be as a post-threshold check. For example, the determined threshold can be compared against the control signal and if the determined threshold significantly exceeds the control signal, it can be determined that the threshold is in error and feedback regarding the same can be output. If the threshold is determined to be in error, then a reanalysis or manual operation can occur based on feedback provided to a user.

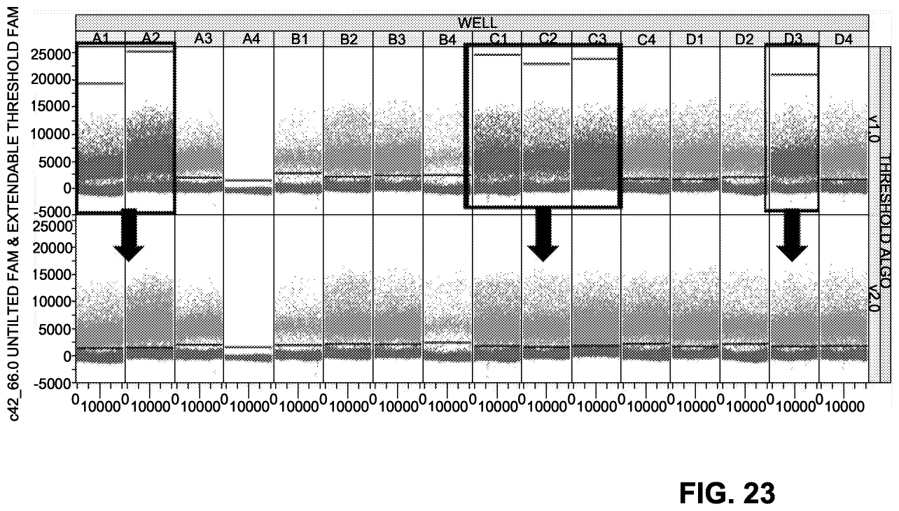

Referring to FIG. 23, example data sets are illustrated that reflect dense clustering with poor separation and “rainy” data. In other words, as shown, the clusters in the data sets outlined in the three boxes contain dense and close together (poorly separated) clusters, as well as data at higher signals that is rainy. Such data sets can be challenging for the thresholding techniques based on the separation (emptiness between clusters. As can be seen in the top data sets, without the use of a control signal, the threshold determination according to the techniques described herein did not perform accurately, placing the threshold generally above all of the data as shown by the horizontal black lines in the top data sets. The corresponding data sets in the bottom row (shown by the arrows from the top to the bottom data sets) show the placement of the threshold (horizontal black line) when the algorithm is input with the positive control signal as described above. These data sets reflect the improved accuracy in the thresholding, resulting in the determination that the dense data actually represents a positive and a negative cluster.

The above uses of a control signal are not mutually exclusive and can be implemented together. Moreover, the numerical values selected, such as for soft threshold and/or bin size, are by way of example and are not limiting with other values chosen based on factors such as the number of data points, particular assays, signal strength (e.g., intensity), etc.

### Adaptive Thresholding

Various other implementations for automated determination of a threshold for dPCR assays can include adaptive techniques that utilize a control signal to predictively adapt thresholding parameters based on the number of positive data points anticipated. For example, a soft threshold as described above can be used to determine the number of positive datapoints within a data set. In datasets which are anticipated to contain a greater number of positive datapoints, the maximum quantile of the search range R is decreased. In a dataset that contains close to 50% positive and negative datapoints, the final threshold placement will most likely be closer to the median signal level of the dataset. Therefore, searching a wider signal level such as between the 1% and 99% quantile will be less desirable than searching a narrower signal level such as between the 5% to 95% quantile.

In addition to decreasing the maximum quantile of the search range R, the number of datapoints used in the threshold determination, in other words the total range R, can be decreased and/or the bin size can be decreased. This reduces the entire distribution so as to increase the number of viable bins to place a threshold.

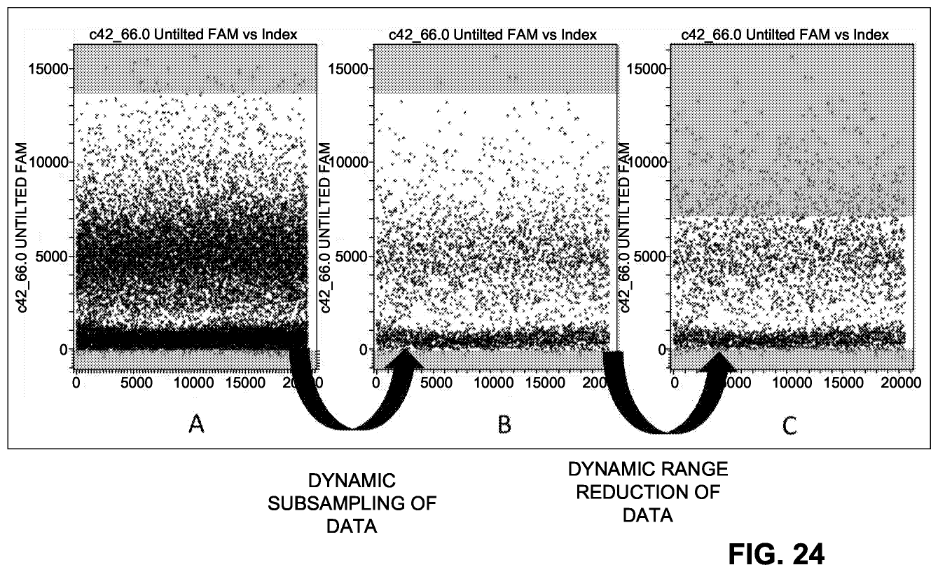

In addition to the above, data may be dynamically subsampled to provide data that is easier for the thresholding techniques described herein to utilize to determine the thresholding. Based on the anticipated number of positives, the data can be subsampled to reduce the overall number of data points while still maintaining a similar distribution of the data. This is reflected in FIG. 24, moving from data set A, (the entire raw data set) to data set B (the sub-sampled data set). This can be done by correlating the proportionality of positives to negatives to the proportion of how many datapoints are sampled out of the total dataset. A data set that is 50% positive may require only half or a tenth of all datapoints to be able to accurately estimate a threshold. A data set that is 5% positive or 5% negative would conversely require a majority of all datapoints to be sampled to ensure accurate thresholding. In addition to the dynamic overall sub-sampling of the data, an additional dynamic range reduction can occur as discussed above and reflected from data set B to data set C where data exceeding the 95% quantile is excluded from the data set being used for thresholding. Again, this reduces the number of data points used in the thresholding determination.

Yet another type of adaptive thresholding can occur through the use of dynamic bin size adjustment. For example, when a determination of a rainy data set has been made, such as through use of a control signal as discussed above, the bin size determined by the initial parameters can be adjusted to enhance the threshold determination by shrinking the bin size when necessary to accommodate for rainier data. In a data set estimated to have a large proportion of positives, the bin size can be reduced to find more viable thresholds, whereas a data set estimated to have few positives can use a relatively larger bin size can be used to reject negative double bands and baseline abnormalities. In one nonlimiting implementation, for example, the bin size could be a set standard bin size for 0% positives in a data set (all negative data set), and the bin size can be cut in half for a sample that has 80% positives; a relationship can be used to set the bin size based on the amount of positives between the two ends, which could be a linear or non-linear relationship.

Overall, using the low concentration, “emptiness” bin techniques described in accordance with the various implementations described herein based on a single population (e.g., negative or positive cluster of data) can provide a robust way of auto-thresholding for use in dPCR assay analysis. In addition, utilizing control signal and adaptive (dynamic) parameter adjustment techniques as described above can further improve the accuracy and/or efficiency in threshold determination. The techniques according to various implementations provide for simpler handling of data of various modalities (normal, bimodal, trimodal, etc.), the ability to more accurately determine a threshold despite imperfect baseline, robust performance in determining thresholds for predominantly negative/saturated/unknown data types. Moreover, various tests utilizing the techniques in accordance with implementations of the present disclosure have provided accurate results for rare allele detection dPCR assays.

### Computer System Implementation

In some embodiments, at least a portion of the methods described herein may be implemented using one or more computer systems. In some instances, the techniques discussed herein are represented in computer-executable instructions that may be stored on one or more hardware storage devices. The computer-executable instructions may be executable by one or more processors to carry out (or to configure a system to carry out) the disclosed techniques. In some embodiments, a system may be configured to send the computer-executable instructions to a remote device to configure the remote device for carrying out the disclosed techniques.

In an example embodiment, a computer system comprises one or more processors, and a memory storing one or more instructions which, when executed by the one or more processors, cause the one or more processors to perform a process such as one or more actions of the workflows 500, 700, 800, 900, 1100, and/or 1200.

Some embodiments include one or more computer-readable media storing one or more instructions which, when executed by one or more processors of at least one computing device, cause the one or more processors to perform the foregoing process or other computer-implemented process as described herein.

Systems for implementing the disclosed embodiments may include various components, such as, by way of non-limiting example, processor(s), storage, sensor(s), I/O system(s), communication system(s), and the like. The processor(s) may comprise one or more sets of electronic circuitries that include any number of logic units, registers, and/or control units to facilitate the execution of computer-readable instructions (e.g., instructions that form a computer program). Such computer-readable instructions may be stored within storage. The storage may comprise physical system memory and may be volatile, non-volatile, or some combination thereof. Furthermore, storage may comprise local storage, remote storage (e.g., accessible via communication system(s) or otherwise), or some combination thereof.

Furthermore, a system may comprise or be in communication with I/O system(s). I/O system(s) may include any type of input or output device such as, by way of non-limiting example, a touch screen, a mouse, a keyboard, a controller, a speaker and/or others, without limitation. For example, the I/O system(s) may include a display system that may comprise any number of display panels, optics, laser scanning display assemblies, and/or other components.

Disclosed embodiments may also include physical and other computer-readable media for carrying or storing computer-executable instructions and/or data structures.

Such computer-readable media can be any available media that can be accessed by a general-purpose or special-purpose computer system. Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

Those skilled in the art will appreciate that various aspects of the workflows and/or techniques described herein may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

Those of ordinary skill in the art will also appreciate that any feature or operation disclosed herein may be combined with any one or combination of the other features and operations disclosed herein. Additionally, the content or feature in any one of the figures may be combined or used in connection with any content or feature used in any of the other figures. In this regard, the content disclosed in any one figure is not mutually exclusive and instead may be combinable with the content from any of the other figures. Moreover, the various actions of the workflows disclosed can be used in any combination and a workflow need not include all actions as depicted in the figures.

The described embodiments are to be considered in all respects only as illustrative and not restrictive. All changes which come within the meaning and range of equivalency of the claims are to be embraced within their scope.

1.31. (canceled)

32. A method for analyzing emission signal data from a digital PCR (dPCR) assay, the method comprising:
generating a histogram from emission data collected from reaction sites of a dPCR assay, the histogram comprising a plurality of bins representing a number of reaction sites at different levels of emission signal;
identifying a continuous range of bins, wherein each bin in the continuous range has a number of reaction sites per bin less than or equal to a predetermined low concentration of number of reaction sites per bin;
selecting a bin from the continuous range of bins;
setting a threshold emission signal level as an emission signal level in the selected bin; and
for a reaction site, classifying emission signal data from the reaction site as positive for amplification of target from the dPCR assay if a level of emission signal corresponding to the reaction site is above the threshold emission signal level or classifying the emission signal data from the reaction site as negative for amplification of the dPCR assay if the level of emission signal corresponding to the reaction site is below the threshold emission signal level.

33. The method of claim 32, wherein selecting the bin from the continuous range of bins comprises selecting a bin corresponding to a highest level of emission signal in the continuous range of bins.

34. The method of claim 33, wherein selecting the bin comprises selecting a bin adjacent to a cluster of bins corresponding to a range of emission signal levels indicating positive for amplification product from the dPCR assay.

35. The method of claim 32, wherein selecting the bin from the continuous range of bins comprises selecting a bin corresponding to a lowest level of emission signal in the continuous range of bins.

36. The method of claim 35, wherein selecting the bin comprises selecting a bin adjacent to a cluster of bins corresponding to a range of emission signal levels indicating negative for amplification product from the dPCR assay.

37. The method of claim 32, further comprising:
identifying a first continuous range of bins and setting a first threshold emission signal level estimate based on an emission signal level associated with a first selected bin from the first continuous range; and
setting a second continuous range of bins narrower than the first continuous range and setting a second threshold emission signal level estimate based on an emission signal level associated with a second selected bin from the second continuous range of bins,
wherein selecting the bin and setting the threshold comprise selecting one of the first or second bins and the corresponding first or second threshold emission signal level estimates.

38. The method of claim 37, wherein the first continuous range comprises bins having a number of reaction sites per bin less than or equal to 0.05% to 10% of the total number of data points in all the bins of the histogram.

39. The method of claim 38, wherein the continuous range of bins comprise bins having a number of reaction sites per bin less than or equal to 0.1% of the total number of data points in all the bins of the histogram.

40. The method of claim 32, further comprising setting a bin size for each of the plurality of bins.

41. The method of claim 40, wherein setting the bin size comprises setting the bin size based on expected emission signal level associated with a reaction volume being positive for amplification product.

42. The method of claim 41, wherein setting the bin size comprises setting the bin size such that each bin is a predetermined percentage of the expected emission signal level.

43. The method of claim 42, wherein the predetermined percentage ranges from 0.1% to 10 %.

44. The method of claim 41, wherein the expected emission signal level is based on a positive control signal.

45. The method of claim 40, wherein setting the bin size comprises setting the bin size based on applying Sturges formula.

46. A method for analyzing emission signal data from a digital PCR (dPCR) assay, the method comprising:
generating a histogram from emission data collected from reaction sites of a dPCR assay, the histogram comprising a plurality of bins representing a number of reaction sites at different levels of emission signal;
identifying a first subset of the plurality of bins, the first subset comprising bins having low concentration based on a number of reaction sites per bin being less than or equal to a predetermined amount;
identifying a first bin in the first subset of bins that corresponds to a highest level of emission signal;
setting a first threshold estimate of emission signal level at the highest level of emission signal of the identified first bin;
identifying a second subset of the plurality of bins adjacent to and over a range of emission signal level lower than the first threshold estimate of emission signal level;
identifying a second bin in the second subset of bins that corresponds to a lowest level of emission signal;
setting a second threshold estimate of emission signal level at the lowest level of emission signal of the identified second bin;
comparing the population of data below the first threshold estimate to the population of data below the second threshold estimate;
based on the comparing, setting a threshold level for classification of the emission signal data as either the first threshold estimate or the second threshold estimate;
for a reaction site, classifying emission signal data from the reaction site as positive for amplification of target from the dPCR assay if the level of emission signal corresponding to the reaction site is above the threshold level or classifying the emission signal data as negative for amplification of target from the dPCR assay if the level of emission signal corresponding to the reaction site is below the threshold level.
